SEQUENCE LISTING (14 sequences)

SEQ ID NO: 1 - DNA, 1263 bp, Corynebacterium glutamicum
  CDS (1)..(1263): lysC wild-type gene
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgc

SEQ ID NO: 2 - protein, 421 aa, Corynebacterium glutamicum
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 3 - DNA, 1263 bp, Corynebacterium glutamicum
  CDS (1)..(1263): lysC-fbr allele lysC T311I
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcatcttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgc

SEQ ID NO: 4 - protein, 421 aa, Corynebacterium glutamicum
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDIIFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 5 - DNA, 20 bp, Corynebacterium glutamicum
  misc_feature (1)..(20): Primer lysCK1
tcggtgtcatcagagcattg

SEQ ID NO: 6 - DNA, 20 bp, Corynebacterium glutamicum
  misc_feature (1)..(20): Primer lysCK2
tcggttgcctgagtaatgtc

SEQ ID NO: 7 - DNA, 20 bp, Corynebacterium glutamicum
  misc_feature (1)..(20): LC-lysC1-fbr
aaccgttctgggtatttccg

SEQ ID NO: 8 - DNA, 20 bp, Corynebacterium glutamicum
  misc_feature (1)..(20): LC-lysC2-fbr
tccatgaactctgcggtaac

SEQ ID NO: 9 - DNA, 21 bp, Corynebacterium glutamicum
  misc_feature (1)..(21): oligonucleotide lysC311-C
gcaggtgaagatgatgtcggt

SEQ ID NO: 10 - DNA, 35 bp, Corynebacterium glutamicum
  misc_feature (1)..(35): Oligonukleotid lysC311-A
tcaagatctccatcgcgcggcggccgtcggaacga

SEQ ID NO: 11 - DNA, 20 bp, Corynebacterium glutamicum
  misc_feature (1)..(20): Primer lysEK-1
tgcttgcacaaggacttcac

SEQ ID NO: 12 - DNA, 20 bp, Corynebacterium glutamicum
  misc_feature (1)..(20): Primer lysEK-2
tatggtccgcaagctcaatg

SEQ ID NO: 13 - DNA, 20 bp, Corynebacterium glutamicum
  misc_feature (1)..(20): Primer zwa1-A2
cacttgtcctcaccactttc

SEQ ID NO: 14 - DNA, 20 bp, Corynebacterium glutamicum
  misc_feature (1)..(20): Primer zwa1-E1
ttctactgggcgtactttcg

(19) United States
(12) Patent Application Publication
(10) Pub. No.: US 2010/0255544 A1
(43) Pub. Date: Oct. 7, 2010

(54) CORYNEFORM BACTERIA WHICH PRODUCE CHEMICAL COMPOUNDS II

(75) Inventors: Brigitte Bathe, Salzkotten (DE); Caroline Kreutzer, Melle (DE); Bettina Mockel, Dusseldorf (DE); Georg Thierbach, Bielefeld (DE)

Correspondence Address: PILLSBURY WINTHROP SHAW PITTMAN, LLP, P.O. BOX 10500, MCLEAN, VA, 22102, US

(73) Assignee: Evonik Degussa GmbH, Essen (DE)

(21) Appl. No.: 12/553,647

(22) Filed: Sep. 3, 2009

Related U.S. Application Data
(63) Continuation of application No. 10/358,393, filed on Feb. 5, 2003, now abandoned.
Application No. 10/358,393 is a continuation of application No. PCT/EP02/08465, filed on Jul. 30, 2002.
(60) Provisional application No. 60/309,877, filed on Aug. 6, 2001.

(51) Int. Cl. C12P 19/38; C12P 1/00; C12P 19/30; C12P 13/04; C12P 13/24; C12P 13/22; C12P 13/20; C12P 13/14; C12P 13/12; C12P 13/08; C12P 13/06; C12N 15/74; C12N 1/21; C12N 15/63

(52) U.S. Cl. 435/87; 435/41; 435/89; 435/106; 435/107; 435/108; 435/109; 435/110; 435/113; 435/115; 435/116; 435/471; 435/252.32; 435/320.1

(57) ABSTRACT

The invention relates to coryneform bacteria which, instead of the singular copy of an open reading frame (ORF), gene or allele naturally present at the particular desired site (locus), have at least two copies of the open reading frame (ORF), gene or allele in question, preferably in tandem arrangement, and optionally at least a third copy of the open reading frame (ORF), gene or allele in question at a further gene site, and processes for the preparation of chemical compounds by fermentation of these bacteria.

This is a continuation of International Patent Appl. No. PCT/EP02/08465, filed Jul. 30, 2002, which claims priority to U.S. Prov. Appl. No. 60/309,877, filed Aug. 6, 2001.

### BACKGROUND

Chemical compounds, which means, in particular, L-amino acids, vitamins, nucleosides and nucleotides and D-amino acids, are used in human medicine, in the pharmaceuticals industry, in cosmetics, in the foodstuffs industry and in animal nutrition.

Numerous of these compounds are prepared by fermentation from strains of coryneform bacteria, in particular Corynebacterium glutamicum. Because of their great importance, work is constantly being undertaken to improve the preparation processes. Improvements to the process can relate to fermentation measures, such as, for example, stirring and supply of oxygen, or the composition of the nutrient media, such as, for example, the sugar concentration during the fermentation, or the working up to the product form by, for example, ion exchange chromatography, or the intrinsic output properties of the microorganism itself.

Methods of mutagenesis, selection and mutant selection are used to improve the output properties of these microorganisms. Strains which are resistant to antimetabolites or are auxotrophic for metabolites of regulatory importance and which produce the particular compounds are obtained in this manner.

Methods of the recombinant DNA technique have also been employed for some years for improving the strain of Corynebacterium strains, by amplifying individual biosynthesis genes and investigating the effect on production.

A common method comprises amplification of certain biosynthesis genes in the particular microorganism by means of episomally replicating plasmids. This procedure has the disadvantage that during the fermentation, which in industrial processes is in general associated with numerous generations, the plasmids are lost spontaneously (segregational instability).

Another method comprises duplicating certain biosynthesis genes by means of plasmids which do not replicate in the particular microorganism. In this method, the plasmid, including the cloned biosynthesis gene, is integrated into the chromosomal biosynthesis gene of the microorganism (Reinscheid et al., Applied and Environmental Microbiology 60(1), 126-132 (1994); Jetten et al., Applied Microbiology and Biotechnology 43(1):76-82 (1995)). A disadvantage of this method is that the nucleotide sequences of the plasmid and of the antibiotic resistance gene necessary for the selection remain in the microorganism. This is a disadvantage, for example, for the disposal and utilization of the biomass. Moreover, the expert expects such strains to be unstable as a result of disintegration by “Campbell type cross over” in a corresponding number of generations such as are usual in industrial fermentations.

### OBJECT OF THE INVENTION

The inventors had the object of providing new measures for improved fermentative preparation of chemical compounds using coryneform bacteria.

### SUMMARY OF THE INVENTION

The invention provides coryneform bacteria, in particular of the genus Corynebacterium, which produce one or more desired chemical compounds, characterized in that
a) instead of the singular copy of an open reading frame (ORF), gene or allele naturally present at the particular desired site (locus), these have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
b) optionally have at least a third copy of the open reading frame (ORF), gene or allele in question at a further gene site, no nucleotide sequence which is capable of/enables episomal replication in microorganisms, no nucleotide sequence which is capable of/enables transposition and no nucleotide sequence which imparts resistance to antibiotics being present at the further gene site.

The invention also provides processes for the preparation of one or more chemical compounds, which comprise the following steps:
a) fermentation of coryneform bacteria, in particular of the genus Corynebacterium, which
   i) instead of the singular copy of an open reading frame (ORF), gene or allele naturally present at the particular desired site (locus), have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
   ii) optionally have at least a third copy of the said open reading frame (ORF), gene or allele at a further gene site, no nucleotide sequence which is capable of/enables episomal replication in microorganisms, no nucleotide sequence which is capable of/enables transposition and no nucleotide sequence which imparts resistance to antibiotics being present at the further gene site,
under conditions which allow expression of the said open reading frames (ORFs) genes or alleles,
b) concentration of the chemical compound(s) in the fermentation broth and/or in the cells of the bacteria,
c) isolation of the chemical compound(s), optionally
d) with constituents from the fermentation broth and/or the biomass to the extent of >(greater than) 0 to 100%.

### DETAILED DESCRIPTION OF THE INVENTION

Chemical compounds are to be understood, in particular, as meaning amino acids, vitamins, nucleosides and nucleotides. The biosynthesis pathways of these compounds are known and are available in the prior art.

Amino acids mean, preferably, L-amino acids, in particular the proteinogenic L-amino acids, chosen from the group consisting of L-aspartic acid, L-asparagine, L-threonine, L-serine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine and salts thereof, in particular L-lysine, L-methionine and L-threonine. L-Lysine is very particularly preferred.

Proteinogenic amino acids are understood as meaning the amino acids which occur in natural proteins, that is to say in proteins of microorganisms, plants, animals and humans.

Vitamins mean, in particular, vitamin B1 (thiamine), vitamin B2 (riboflavin), vitamin B5 (pantothenic acid), vitamin B6 (pyridoxines), vitamin B12 (cyanocobalamin), nicotinic acid/nicotinamide, vitamin M (folic acid) and vitamin E (tocopherol) and salts thereof, pantothenic acid being preferred.

Nucleosides and nucleotides mean, inter alia, S-adenosyl-methionine, inosine-5′-monophosphoric acid and guanosine-5′-monophosphoric acid and salts thereof.

The coryneform bacteria are, in particular, those of the genus Corynebacterium. Of the genus Corynebacterium, the species Corynebacterium glutamicum, Corynebacterium ammoniagenes and Corynebacterium thermoaminogenes are preferred. Information on the taxonomic classification of strains of this group of bacteria is to be found, inter alia, in Kämpfer and Kroppenstedt (Canadian Journal of Microbiology 42, 989-1005 (1996)) and in U.S. Pat. No. 5,250,434.

Suitable strains of the species Corynebacterium glutamicum (C. glutamicum) are, in particular, the known wild-type strains
Corynebacterium glutamicum ATCC13032
Corynebacterium acetoglutamicum ATCC15806
Corynebacterium acetoacidophilum ATCC13870
Corynebacterium lilium ATCC15990
Corynebacterium melassecola ATCC17965
Corynebacterium herculis ATCC13868
Arthrobacter sp ATCC243

Brevibacterium chang-fua ATCC14017
Brevibacterium flavum ATCC14067
Brevibacterium lactofermentum ATCC13869
Brevibacterium divaricatum ATCC14020
Brevibacterium taipei ATCC13744 and
Microbacterium ammoniaphilum ATCC21645
   and mutants or strains, such as are known from the prior art, produced therefrom which produce chemical compounds.

Suitable strains of the species Corynebacterium ammoniagenes (C. ammoniagenes) are, in particular, the known wild-type strains
Brevibacterium ammoniagenes ATCC6871
Brevibacterium ammoniagenes ATCC15137 and
Corynebacterium sp. ATCC21084
   and mutants or strains, such as are known from the prior art, produced therefrom which produce chemical compounds.

Suitable strains of the species Corynebacterium thermoaminogenes (C. thermoaminogenes) are, in particular, the known wild-type strains
Corynebacterium thermoaminogenes FERM BP-1539
Corynebacterium thermoaminogenes FERM BP-1540
Corynebacterium thermoaminogenes FERM BP-1541 and
Corynebacterium thermoaminogenes FERM BP-1542
   and mutants or strains, such as are known from the prior art, produced therefrom which produce chemical compounds.

Strains with the designation “ATCC” can be obtained from the American Type Culture Collection (Manassas, Va., USA). Strains with the designation “FERM” can be obtained from the National Institute of Advanced Industrial Science and Technology (AIST Tsukuba Central 6, 1-1-1 Higashi, Tsukuba Ibaraki, Japan). The strains of Corynebacterium thermoaminogenes mentioned (FERM BP-1539, FERM BP-1540, FERM BP-1541 and FERM BP-1542) are described in U.S. Pat. No. 5,250,434.

Open reading frame (ORF) describes a section of a nucleotide sequence which codes or can code for a protein or polypeptide or ribonucleic acid to which no function can be assigned according to the prior art.

After assignment of a function to the nucleotide sequence section in question, it is in general referred to as a gene.

Alleles are in general understood as meaning alternative forms of a given gene. The forms are distinguished by differences in the nucleotide sequence.

In the context of the present invention, endogenous, that is to say species-characteristic, open reading frames, genes or alleles are preferably used. These are understood as meaning the open reading frames, genes or alleles or nucleotide sequences thereof present in the population of a species, such as, for example, Corynebacterium glutamicum.

A “singular copy of an open reading frame (ORF), gene or allele naturally present at the particular desired site (locus)” is understood as meaning the circumstances that a gene in general naturally occurs in one (1) copy in the form of its nucleotide sequence at its site or gene site in the corresponding wild-type or corresponding parent organism or starting organism. This site is preferably in the chromosome.

Thus, for example, the lysC gene or an lysC^{FBR} allele which codes for a “feed back” resistant aspartate kinase is present in one copy at the lysC site or lysC locus or lysC gene site and is flanked by the open reading frame orfX and the leuA gene on one side and by the asd gene on the other side.

“Feed back” resistant aspartokinases are understood as meaning aspartokinases which, compared with the wild-type form, have a lower sensitivity to inhibition by mixtures of lysine and threonine or mixtures of AEC (aminoethylcysteine) and threonine or lysine by itself or AEC by itself. Strains which produce L-lysine typically contain such “feed back” resistant or desensitized aspartokinases.

The nucleotide sequence of the chromosome of Corynebacterium glutamicum is known and can be found in the patent application EP-A-1108790 and Access Number (Accession No.) AX114121 of the nucleotide sequence databank of the European Molecular Biologies Laboratories (EMBL, Heidelberg, Germany and Cambridge, UK). The nucleotide sequences of orfX, the leuA gene and the asd gene have the Access Numbers AX120364 (orfX), AX123517 (leuA) and AX123519 (asd).

Further databanks, such as, for example, that of the National Center for Biotechnology Information (NCBI, Bethesda, Md., USA) or that of the Swiss Institute of Bioinformatics (Swissprot, Geneva, Switzerland) or that of the Protein Information Resource Database (PIR, Washington, D.C., USA) can also be used.

“Tandem arrangement” of two or more copies of an open reading frame (ORF), gene or allele is referred to if these are arranged in a row directly adjacent in the same orientation.

“A further gene site” is understood as meaning a second gene site, the nucleotide sequence of which is different from the sequence of the ORF, gene or allele which has been at least duplicated at the natural site. This further gene site, or the nucleotide sequence present at the further gene site, is preferably in the chromosome and is in general not essential for growth and for production of the desired chemical compounds.

The “further gene sites” mentioned include, of course, not only the coding regions of the open reading frames or genes mentioned, but also the regions or nucleotide sequences lying upstream which are responsible for expression and regulation, such as, for example, ribosome binding sites, promoters, binding sites for regulatory proteins, binding sites for regulatory ribonucleic acids and attenuators. These regions in general lie in a range of 1-800, 1-600, 1-400, 1-200, 1-100 or 1-50 nucleotides upstream of the coding region. In the same way, regions lying downstream, such as, for example, transcription terminators, are also included. These regions in general lie in a range of 1-400, 1-200, 1-100, 1-50 or 1-25 nucleotides downstream of the coding region.

Intergenic regions in the chromosome, that is to say nucleotide sequences without a coding function, can furthermore be used. Finally, prophages or defective phages or DNA coding for phage components contained in the chromosome can be used for this.

Examples of regions of the Corynebacterium glutamicum chromosome representing intergenic regions, prophages, defective phages or phage components are shown in tables 12 and 13. The positions of the DNA regions refer to the genome map of Corynebacterium glutamicum ATCC 13032 as presented in EP-A-1108790 or in the databank of the European Molecular Biologies Laboratories (EMBL, Heidelberg, Germany and Cambridge, UK).

A prophage is understood as meaning a bacteriophage, in particular the genome thereof, where this is replicated together with the genome of the host and the formation of infectious particles does not take place. A defective phage is understood as meaning a prophage, in particular the genome thereof, which, as a result of various mutations, has lost the ability to form so-called infectious particles. Defective phages are also called cryptic.

Prophages and defective phages are often present in integrated form in the chromosome of their host. Further details exist in the prior art, for example in the textbook by Edward A. Birge (Bacterial and Bacteriophage Genetics, 3^{rd} ed., Springer-Verlag, New York, USA, 1994) or in the textbook by S. Klaus et al. (Bakterienviren, Gustav Fischer Verlag, Jena, Germany, 1992).

To produce the coryneform bacteria according to the invention, the nucleotide sequence of the desired ORF, gene or allele, preferably including the expression and/or regulation signals, is isolated, at least two copies are arranged in a row, preferably in tandem arrangement, these are then transferred into the desired coryneform bacterium, preferably with the aid of vectors which do not replicate or replicate to only a limited extent in coryneform bacteria, and those bacteria in which two copies of the ORF, gene or allele are incorporated at the particular desired natural site instead of the singular copy originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus).

The expression and/or regulation signals mentioned, such as, for example, the ribosome binding sites, promoters, binding sites for regulatory proteins, binding sites for regulatory ribonucleic acids and attenuators lying upstream of the coding region of the ORF, gene or allele, are in general in a range of 1-800, 1-600, 1-400, 1-200, 1-100 or 1-50 nucleotides upstream of the coding region. The expression and/or regulation signals mentioned, such as, for example, the transcription terminators lying downstream of the coding region of the ORF, gene or allele, are in general in a range of 1-400, 1-200, 1-100, 1-50 or 1-25 nucleotides downstream of the coding region.

Preferably, also, no residues of sequences of the vectors used or species-foreign DNA, such as, for example, restriction cleavage sites, remain on the flanks of the ORFs, genes or alleles amplified according to the invention. In each case a maximum of 24, preferably a maximum of 12, particularly preferably a maximum of 6 nucleotides of such DNA optionally remain on the flanks.

At least a third copy of the open reading frame (ORF), gene or allele in question is optionally inserted at a further gene site, or several further gene sites, no nucleotide sequence which is capable of/enables episomal replication in microorganisms, no nucleotide sequence which is capable of/enables transposition and no nucleotide sequence which imparts resistance to antibiotics being present at the further gene site.

Preferably, also, no residues of sequences of the vectors used or species-foreign DNA, such as, for example, restriction cleavage sites, remain at the further gene site. A maximum of 24, preferably a maximum of 12, particularly preferably a maximum of 6 nucleotides of such DNA upstream or downstream of the ORF, gene or allele incorporated optionally remain at the further gene site.

The invention accordingly also provides a process for the production of coryneform bacteria which produce one or more chemical compounds, characterized in that
a) the nucleotide sequence of a desired ORF, gene or allele, preferably including the expression and/or regulation signals, is isolated
b) at least two copies of the nucleotide sequence of the ORF, gene or allele are arranged in a row, preferably in tandem arrangement
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired ORF, gene or allele at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and
f) at least a third copy of the open reading frame (ORF), gene or allele in question is optionally introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

By the measures according to the invention, the productivity of the coryneform bacteria or of the fermentative processes for the preparation of chemical compounds is improved in respect of one or more of the features chosen from the group consisting of concentration (chemical compound formed, based on the unit volume), yield (chemical compound formed, based on the source of carbon consumed) and product formation rate (chemical compound formed, based on the time) by at least 0.5-1.0% or at least 1.0 to 1.5% or at least 1.5-2.0%.

Instructions on conventional genetic engineering methods, such as, for example, isolation of chromosomal DNA, plasmid DNA, handling of restriction enzymes etc., are found in Sambrook et al. (Molecular Cloning—A Laboratory Manual (1989) Cold Spring Harbor Laboratory Press). Instructions on transformation and conjugation in coryneform bacteria are found, inter alia, in Thierbach et al. (Applied Microbiology and Biotechnology 29, 356-362 (1988)), in Schäfer et al. (Journal of Bacteriology 172, 1663-1666 (1990) and Gene 145, 69-73 (1994)) and in Schwarzer and Pühler (Bio/Technology 9, 84-87 (1991)).

Vectors which replicate to only a limited extent are understood as meaning plasmid vectors which, as a function of the conditions under which the host or carrier is cultured, replicate or do not replicate. Thus, a temperature-sensitive plasmid for coryneform bacteria which can replicate only at temperatures below 31° C. has been described by Nakamura et al. (U.S. Pat. No. 6,303,383).

The invention also provides coryneform bacteria, in particular of the genus Corynebacterium, which produce L-lysine, characterized in that
a) instead of the singular copy of an open reading frame (ORF), a gene or allele of lysine production naturally present at the particular desired site (locus), these have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
b) optionally have at least a third copy of the said open reading frame (ORF), gene or allele of L-lysine production at a further gene site, no nucleotide sequence which is capable of/enables episomal replication in microorganisms, no nucleotide sequence which is capable of/enables transposition and no nucleotide sequence which imparts resistance to antibiotics being present at the further gene site.

The invention also furthermore provides a process for the preparation of L-lysine, which comprises the following steps:
a) fermentation of coryneform bacteria, in particular of the genus Corynebacterium, which
   i) instead of the singular copy of an open reading frame (ORF), gene or allele of lysine production present at the particular desired site (locus), have at least two copies of the open reading frame (ORF), gene or allele in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
   ii) optionally have at least a third copy of the open reading frame (ORF), gene or allele of L-lysine production in question at a further gene site, no nucleotide sequence which is capable of/enables episomal replication in microorganisms, no nucleotide sequence which is capable of/enables transposition and no nucleotide sequence which imparts resistance to antibiotics being present at the further gene site,
      under conditions which allow expression of the said open reading frames (ORFs), genes or alleles,
b) concentration of the L-lysine in the fermentation broth,
c) isolation of the L-lysine from the fermentation broth, optionally
d) with constituents from the fermentation broth and/or the biomass to the extent of >(greater than) 0 to 100%.

A “copy of an open reading frame (ORF), gene or allele of lysine production” is to be understood as meaning all the, preferably endogenous, open reading frames, genes or alleles of which enhancement/over-expression can have the effect of improving lysine production. Enhancement is understood as meaning an increase in the intracellular concentration or activity of the particular gene product, protein or enzyme.

These include, inter alia, the following open reading frames, genes or alleles: accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysC^{FBR}, lysE, msiK, opcA, oxyR, ppc, ppc^{FBR}, pgk, pknA, pknB, pknD, pknG, ppsA, ptsH, ptsI, ptsM, pyc, pyc P458S, sigC, sigD, sigE, sigH, sigM, tal, thyA, tkt, tpi, zwa1, zwf and zwf A213T. These are summarized and explained in Table 1.

These include, in particular, the lysC^{FBR} alleles which code for a “feed back” resistant aspartate kinase. Various lysC^{FBR} alleles are summarized and are explained in Table 2.

The following lysC^{FBR} alleles are preferred: lysC A279T (replacement of alanine at position 279 of the aspartate kinase protein coded, according to SEQ ID NO: 2, by threonine), lysC A279V (replacement of alanine at position 279 of the aspartate kinase protein coded, according to SEQ ID NO: 2, by valine), lysC S301F (replacement of serine at position 301 of the aspartate kinase protein coded, according to SEQ ID NO: 2, by phenylalanine), lysC T308I (replacement of threonine at position 308 of the aspartate kinase protein coded, according to SEQ ID NO: 2, by isoleucine), lysC S301Y (replacement of serine at position 308 of the aspartate kinase protein coded, according to SEQ ID NO: 2, by tyrosine), lysC G345D (replacement of glycine at position 345 of the aspartate kinase protein coded, according to SEQ ID NO: 2, by aspartic acid), lysC R320G (replacement of arginine at position 320 of the aspartate kinase protein coded, according to SEQ ID NO: 2, by glycine), lysC T311I (replacement of threonine at position 311 of the aspartate kinase protein coded, according to SEQ ID NO: 2, by isoleucine), lysC S381F (replacement of serine at position 381 of the aspartate kinase protein coded, according to SEQ ID NO: 2, by phenylalanine).

The lysC^{FBR} allele lysC T311I (replacement of threonine at position 311 of the aspartate kinase protein coded, according to SEQ ID NO: 2, by isoleucine), the nucleotide sequence of which is shown as SEQ ID NO:3, is particularly preferred; the amino acid sequence of the aspartate kinase protein coded is shown as SEQ ID NO:4.

The following open reading frames, genes or nucleotide sequences, inter alia, can be used as the “further gene site” which is not essential for growth or lysine production: aecD, ccpA1, ccpA2, citA, citB, citE, fda, gluA, gluB, gluC, gluD, luxR, luxS, lysR1, lysR2, lysR3, menE, mqo, pck, pgi, poxB and zwa2, in particular the genes aecD, gluA, gluB, gluC, gluD and pck. These are summarized and explained in Table 3. Intergenic regions in the chromosome, that is to say nucleotide sequences without a coding function, can furthermore be used. Finally, prophages or defective phages or DNA coding for phage components contained in the chromosome can be used.

TABLE 1
Open reading frames, genes and alleles of lysine production

| | Description of the coded | | Access |
|---|---|---|---|
| Name | enzyme or protein | Reference | Number |
| accBC | Acyl-CoA Carboxylase | Jäger | U35023 |
| | EC 6.3.4.14 | et al. | AX123524 |
| | (acyl-CoA carboxylase) | Archives of | AX066441 |
| | | Microbiology | |
| | | (1996) 166: 76- | |
| | | 82 | |
| | | EP1108790; | |
| | | WO0100805 | |
| accDA | Acetyl-CoA Carboxylase | EP1055725 | AX121013 |
| | EC 6.4.1.2 | EP1108790 | AX066443 |
| | (acetyl-CoA carboxylase) | WO0100805 | |
| cstA | Carbon Starvation Protein A | EP1108790 | AX120811 |
| | (carbon starvation protein A) | WO0100804 | AX066109 |
| cysD | Sulfate Adenylyltransferase | EP1108790 | AX123177 |
| | sub-unit II | | |
| | EC 2.7.7.4 | | |
| | (sulfate adenylyltransferase | | |
| | small chain) | | |
| cysE | Serine Acetyltransferase | EP1108790 | AX122902 |
| | EC 2.3.1.30 | WO0100843 | AX063961 |
| | (serine acetyltransferase) | | |
| cysH | 3′-Phosphoadenyl Sulfate Reductase | EP1108790 | AX123178 |
| | EC 1.8.99.4 | WO0100842 | AX066001 |
| | (3′-phosphoadenosine 5′- | | |
| | phosphosulfate reductase) | | |
| cysK | Cysteine Synthase | EP1108790 | AX122901 |
| | EC 4.2.99.8 | WO0100843 | AX063963 |
| | (Cysteine synthase) | | |
| cysN | Sulfate Adenylyltransferase sub- | EP1108790 | AX123176 |
| | unit I | | AX127152 |
| | EC 2,7.7.4 | | |
| | (sulfate adenylyltransferase) | | |
| cysQ | Transport protein CysQ | EP1108790 | AX127145 |
| | (transporter cysQ) | WO0100805 | AX066423 |
| dapA | Dihydrodipicolinate Synthase | Bonnassie et | X53993 |
| | EC 4.2.1.52 | al. Nucleic | Z21502 |
| | (dihydrodipicolinate synthase) | Acids Research | AX123560 |
| | | 18: 6421 (1990) | AX063773 |
| | | Pisabarro et | |
| | | al., Journal of | |
| | | Bacteriology | |
| | | 175: 2743- | |
| | | 2749 (1993) | |
| | | EP1108790 | |
| | | WO0100805 | |
| | | EP0435132 | |
| | | EP1067192 | |
| | | EP1067193 | |
| dapB | Dihydrodipicolinate Reductase | EP1108790 | AX127149 |
| | EC 1.3.1.26 | WO0100843 | AX063753 |
| | (dihydrodipicolinate reductase) | EP1067192 | AX137723 |
| | | EP1067193 | AX137602 |
| | | Pisabarro et | X67737 |
| | | al., Journal of | Z21502 |
| | | Bacteriology | E16749 |
| | | 175: 2743- | E14520 |
| | | 2749 (1993) | E12773 |
| | | JP1998215883 | E08900 |
| | | JP1997322774 | |
| | | JP1997070291 | |
| | | JP1995075578 | |
| dapC | N-Succinyl Aminoketopimelate | EP1108790 | AX127146 |
| | Transaminase | WO0100843 | AX064219 |
| | EC 2.6.1.17 | EP1136559 | |
| | (N-succinyl diaminopimelate | | |
| | transaminase) | | |
| dapD | Tetrahydrodipicolinate Succinylase | EP1108790 | AX127146 |
| | EC 2.3.1.117 | WO0100843 | AX063757 |
| | (tetrahydrodipicolinate | Wehrmann et al. | AJ004934 |
| | succinylase) | Journal of | |
| | | Bacteriology | |
| | | 180: 3159- | |
| | | 3165 (1998) | |
| dapE | N-Succinyl Diaminopimelate | EP1108790 | AX127146 |
| | Desuccinylase | WO0100843 | AX063749 |
| | EC 3.5.1.18 | Wehrmann et al. | X81379 |
| | (N-succinyl diaminopimelate | Microbiology | |
| | desuccinylase) | 140: 3349-3356 | |
| | | (1994) | |
| dapF | Diaminopimelate Epimerase | EP1108790 | AX127149 |
| | EC 5.1.1.7 | WO0100843 | AX063719 |
| | (diaminopimelate epimerase) | EP1085094 | AX137620 |
| ddh | Diaminopimelate Dehydrogenase | EP1108790 | AX127152 |
| | EC 1.4.1.16 | WO0100843 | AX063759 |
| | (diaminopimelate dehydrogenase) | Ishino et al., | Y00151 |
| | | Nucleic Acids | E14511 |
| | | Research | E05776 |
| | | 15: 3917- | D87976 |
| | | 3917 (1987) | |
| | | JP1997322774 | |
| | | JP1993284970 | |
| | | Kim et al., | |
| | | Journal of | |
| | | Microbiology | |
| | | and | |
| | | Biotechnology | |
| | | 5: 250-256 (1995) | |
| dps | DNA Protection Protein | EP1108790 | AX127153 |
| | (protection during starvation | | |
| | protein) | | |
| eno | Enolase | EP1108790 | AX127146 |
| | EC 4.2.1.11 | WO0100844 | AX064945 |
| | (enolase) | EP1090998 | AX136862 |
| | | Hermann et al., | |
| | | Electrophoresis | |
| | | 19: 3217-3221 | |
| | | (1998) | |
| gap | Glyceraldehyde 3-Phosphate | EP1108790 | AX127148 |
| | Dehydrogenase | WO0100844 | AX064941 |
| | EC 1.2.1.12 | Eikmanns et | X59403 |
| | (glyceraldehyde 3-phosphate | al., Journal of | |
| | dehydrogenase) | Bacteriology | |
| | | 174: 6076- | |
| | | 6086 (1992) | |
| gap2 | Glyceraldehyde 3-Phosphate | EP1108790 | AX127146 |
| | Dehydrogenase | WO0100844 | AX064939 |
| | EC 1.2.1.12 | | |
| | (glyceraldehyde 3-phosphate | | |
| | dehydrogenase 2) | | |
| gdh | Glutamate Dehydrogenase | EP1108790 | AX127150 |
| | EC 1.4.1.4 | WO0100844 | AX063811 |
| | (glutamate dehydrogenase) | Boermann et | X59404 |
| | | al., Molecular | X72855 |
| | | Microbiology | |
| | | 6: 317-326 | |
| | | (1992). | |
| | | Guyonvarch et | |
| | | al. NCBI | |
| gnd | 6-Phosphogluconate Dehydrogenase | EP1108790 | AX127147 |
| | EC 1.1.1.44 | WO0100844 | AX121689 |
| | (6-phosphogluconate dehydrogenase) | | AX065125 |
| lysC | Aspartate Kinase | EP1108790 | AX120365 |
| | EC 2.7.2.4 | WO0100844 | AX063743 |
| | (aspartate kinase) | Kalinowski et | X57226 |
| | | al., Molecular | |
| | | Microbiology | |
| | | 5: 1197-204 | |
| | | (1991) | |
| lysC^{FBR} | Aspartate Kinase feedback | see Table 2 | |
| | resistent (fbr) | | |
| | EC 2.7.2.4 | | |
| | (aspartate kinase fbr) | | |
| lysE | Lysine Exporter | EP1108790 | AX123539 |
| | (lysine exporter protein) | WO0100843 | AX123539 |
| | | Vrljić et al., | X96471 |
| | | Molecular | |
| | | Microbiology | |
| | | 22: 815-826 | |
| | | (1996) | |
| msiK | Sugar Importer | EP1108790 | AX120892 |
| | (multiple sugar import protein) | | |
| opcA | Glucose 6-Phosphate Dehydrogenase | WO0104325 | AX076272 |
| | (subunit of glucose 6-phosphate | | |
| | dehydrogenase) | | |
| oxyR | Transcription Regulator | EP1108790 | AX122198 |
| | (transcriptional regulator) | | AX127149 |
| ppc^{FBR} | Phosphoenol Pyruvate Carboxylase | EP0723011 | |
| | feedback resistent | WO0100852 | |
| | EC 4.1.1.31 | | |
| | (phosphoenol pyruvate carboxylase | | |
| | feedback resistant) | | |
| ppc | Phosphoenol Pyruvate Carboxylase | EP1108790 | AX127148 |
| | EC 4.1.1.31 | O'Reagan et | AX123554 |
| | (phosphoenol pyruvate carboxylase) | al., Gene | M25819 |
| | | 77 (2): 237- | |
| | | 251 (1989) | |
| pgk | Phosphoglycerate Kinase | EP1108790 | AX121838 |
| | EC 2.7.2.3 | WO0100844 | AX127148 |
| | (phosphoglycerate kinase) | Eikmanns, | AX064943 |
| | | Journal of | X59403 |
| | | Bacteriology | |
| | | 174: 6076-6086 | |
| | | (1992) | |
| pknA | Protein Kinase A | EP1108790 | AX120131 |
| | (protein kinase A) | | AX120085 |
| pknB | Protein Kinase B | EP1108790 | AX120130 |
| | (protein kinase B) | | AX120085 |
| pknD | Protein Kinase D | EP1108790 | AX127150 |
| | (protein kinase D) | | AX122469 |
| | | | AX122468 |
| pknG | Protein Kinase G | EP1108790 | AX127152 |
| | (protein kinase G) | | AX123109 |
| ppsA | Phosphoenol Pyruvate Synthase | EP1108790 | AX127144 |
| | EC 2.7.9.2 | | AX120700 |
| | (phosphoenol pyruvate synthase) | | AX122469 |
| ptsH | Phosphotransferase System Protein | EP1108790 | AX122210 |
| | H | WO0100844 | AX127149 |
| | EC 2.7.1.69 | | AX069154 |
| | (phosphotransferase system | | |
| | component H) | | |
| ptsI | Phosphotransferase System Enzyme I | EP1108790 | AX122206 |
| | EC 2.7.3.9 | | AX127149 |
| | (phosphotransferase system enzyme | | |
| | I) | | |
| ptsM | Glucose-specific | Lee et al., | L18874 |
| | Phosphotransferase System Enzyme | FEMS | |
| | II | Microbiology | |
| | EC 2.7.1.69 | Letters 119 (1- | |
| | (glucose phosphotransferase-system | 2): 137-145 | |
| | enzyme II) | (1994) | |
| pyc | Pyruvate Carboxylase | WO9918228 | A97276 |
| | EC 6.4.1.1 | Peters-Wendisch | Y09548 |
| | (pyruvate carboxylase) | et al., | |
| | | Microbiology | |
| | | 144: 915-927 | |
| | | (1998) | |
| pyc | Pyruvate Carboxylase | EP1108790 | |
| P458S | EC 6.4.1.1 | | |
| | (pyruvate carboxylase) | | |
| | amino acid exchange P458S | | |
| sigC | Sigma Factor C | EP1108790 | AX120368 |
| | EC 2.7.7.6 | | AX120085 |
| | (extracytoplasmic function | | |
| | alternative sigma factor C) | | |
| sigD | RNA Polymerase Sigma Factor D | EP1108790 | AX120753 |
| | EC 2.7.7.6 | | AX127144 |
| | (RNA polymerase sigma factor) | | |
| sigE | Sigma Factor E | EP1108790 | AX127146 |
| | EC 2.7.7.6 | | AX121325 |
| | (extracytoplasmic function | | |
| | alternative sigma factor E) | | |
| sigH | Sigma Factor H | EP1108790 | AX127145 |
| | EC 2.7.7.6 | | AX120939 |
| | (sigma factor SigH) | | |
| sigM | Sigma Factor M | EP1108790 | AX123500 |
| | EC 2.7.7.6 | | AX127153 |
| | (sigma factor SigM) | | |
| tal | Transaldolase EC 2.2.1.2 | WO0104325 | AX076272 |
| | (transaldolase) | | |
| thyA | Thymidylate Synthase | EP1108790 | AX121026 |
| | EC 2.1.1.45 | | AX127145 |
| | (thymidylate synthase) | | |
| tkt | Transketolase | Ikeda et al., | AB023377 |
| | EC 2.2.1.1 | NCBI | |
| | (transketolase) | | |
| tpi | Triose Phosphate Isomerase | Eikmanns, | X59403 |
| | EC 5.3.1.1 | Journal of | |
| | (triose phosphate isomerase) | Bacteriology | |
| | | 174: 6076-6086 | |
| | | (1992) | |
| zwa1 | Cell Growth Factor 1 | EP1111062 | AX133781 |
| | (growth factor 1) | | |
| zwf | Glucose 6-Phosphate 1- | EP1108790 | AX127148 |
| | Dehydrogenase | WO0104325 | AX121827 |
| | EC 1.1.1.49 | | AX076272 |
| | (glucose 6-phosphate 1- | | |
| | dehydrogenase) | | |
| zwf | Glucose 6-Phosphate 1- | EP1108790 | |
| A213T | Dehydrogenase EC 1.1.1.49 | | |
| | (glucose 6-phosphate 1- | | |
| | dehydrogenase) | | |
| | amino acid exchange A213T | | |

TABLE 2
lysC^{FBR} alleles which code for feed back resistant aspartate kinases

| Name of the | Amino acid | | Access |
|---|---|---|---|
| allele | replacement | Reference | Number |
| lysC^{FBR}-E05108 | | JP 1993184366-A | E05108 |
| | | (sequence 1) | |
| lysC^{FBR}-E06825 | lysC A279T | JP 1994062866-A | E06825 |
| | | (sequence 1) | |
| lysC^{FBR}-E06826 | lysC A279T | JP 1994062866-A | E06826 |
| | | (sequence 2) | |
| lysC^{FBR}-E06827 | | JP 1994062866-A | E06827 |
| | | (sequence 3) | |
| lysC^{FBR}-E08177 | | JP 1994261766-A | E08177 |
| | | (sequence 1) | |
| lysC^{FBR}-E08178 | lysC A279T | JP 1994261766-A | E08178 |
| | | (sequence 2) | |
| lysC^{FBR}-E08179 | lysC A279V | JP 1994261766-A | E08179 |
| | | (sequence 3) | |
| lysC^{FBR}-E08180 | lysC S301F | JP 1994261766-A | E08180 |
| | | (sequence 4) | |
| lysC^{FBR}-E08181 | lysC T308I | JP 1994261766-A | E08181 |
| | | (sequence 5) | |
| lysC^{FBR}-E08182 | | JP 1994261766-A | E08182 |
| lysC^{FBR}-E12770 | | JP 1997070291-A | E12770 |
| | | (sequence 13) | |
| lysC^{FBR}-E14514 | | JP 1997322774-A | E14514 |
| | | (sequence 9) | |
| lysC^{FBR}-E16352 | | JP 1998165180-A | E16352 |
| | | (sequence 3) | |
| lysC^{FBR}-E16745 | | JP 1998215883-A | E16745 |
| | | (sequence 3) | |
| lysC^{FBR}-E16746 | | JP 1998215883-A | E16746 |
| | | (sequence 4) | |
| lysC^{FBR}-I74588 | | US 5688671-A | I74588 |
| | | (sequence 1) | |
| lysC^{FBR}-I74589 | lysC A279T | US 5688671-A | I74589 |
| | | (sequence 2) | |
| lysC^{FBR}-I74590 | | US 5688671-A | I74590 |
| | | (sequence 7) | |
| lysC^{FBR}-I74591 | lysC A279T | US 5688671-A | I74591 |
| | | (sequence 8) | |
| lysC^{FBR}-I74592 | | US 5688671-A | I74592 |
| | | (sequence 9) | |
| lysC^{FBR}-I74593 | lysC A279T | US 5688671-A | I74593 |
| | | (sequence 10) | |
| lysC^{FBR}-I74594 | | US 5688671-A | I74594 |
| | | (sequence 11) | |
| lysC^{FBR}-I74595 | lysC A279T | US 5688671-A | I74595 |
| | | (sequence 12) | |
| lysC^{FBR}-I74596 | | US 5688671-A | I74596 |
| | | (sequence 13) | |
| lysC^{FBR}-I74597 | lysC A279T | US 5688671-A | I74597 |
| | | (sequence 14) | |
| lysC^{FBR}-X57226 | lysC S301Y | EP0387527 | X57226 |
| | | Kalinowski et | |
| | | al., Molecular | |
| | | and General | |
| | | Genetics | |
| | | 224: 317-324 | |
| | | (1990) | |
| lysC^{FBR}-L16848 | lysC G345D | Follettie and | L16848 |
| | | Sinskey | |
| | | NCBI Nucleotide | |
| | | Database (1990) | |
| lysC^{FBR}-L27125 | lysC R320G | Jetten et al., | L27125 |
| | lysC G345D | Applied | |
| | | Microbiology | |
| | | Biotechnology | |
| | | 43: 76-82 (1995) | |
| lysC^{FBR} | lysC T311I | WO0063388 | |
| | | (sequence 17) | |
| lysC^{FBR} | lysC S301F | U.S. Pat. No. 3732144 | |
| lysC^{FBR} | lysC S381F | | |
| lysC^{FBR} | | JP6261766 | |
| | | (sequence 1) | |
| lysC^{FBR} | lysC A279T | JP6261766 | |
| | | (sequence 2) | |
| lysC^{FBR} | lysC A279V | JP6261766 | |
| | | (sequence 3) | |
| lysC^{FBR} | lysC S301F | JP6261766 | |
| | | (sequence 4) | |
| lysC^{FBR} | lysC T308I | JP6261766 | |
| | | (sequence 5) | |

TABLE 3
Further gene sites for integration of open reading
frames, genes and alleles of lysine production

| Gene | Description of the coded | | Access |
|---|---|---|---|
| name | enzyme or protein | Reference | Number |
| aecD | beta C-S Lyase | Rossol et al., Journal | M89931 |
| | EC 2.6.1.1 | of Bacteriology 174 | |
| | (beta C-S lyase) | (9): 2968-77 (1992) | |
| ccpA1 | Catabolite Control | WO0100844 | AX065267 |
| | Protein | EP1108790 | AX127147 |
| | (catabolite control | | |
| | protein A1) | | |
| ccpA2 | Catabolite Control | WO0100844 | AX065267 |
| | Protein | EP1108790 | AX121594 |
| | (catabolite control | | |
| | protein A2) | | |
| citA | Sensor Kinase CitA | EP1108790 | AX120161 |
| | (sensor kinase CitA) | | |
| citB | Transcription Regulator | EP1108790 | AX120163 |
| | CitB | | |
| | (transcription regulator | | |
| | CitB) | | |
| citE | Citrate Lyase | WO0100844 | AX065421 |
| | EC 4.1.3.6 | EP1108790 | AX127146 |
| | (citrate lyase) | | |
| fda | Fructose Bisphosphate | von der Osten et al., | X17313 |
| | Aldolase | Molecular | |
| | EC 4.1.2.13 | Microbiology 3 (11): | |
| | (fructose 1,6- | 1625-37 (1989) | |
| | bisphosphate aldolase) | | |
| gluA | Glutamate Transport | Kronemeyer et al., | X81191 |
| | ATP-binding Protein | Journal of | |
| | (glutamate transport | Bacteriology 177 (5): | |
| | ATP-binding protein) | 1152-8 (1995) | |
| gluB | Glutamate-binding | Kronemeyer et al., | X81191 |
| | Protein | Journal of | |
| | (glutamate-binding | Bacteriology 177 (5): | |
| | protein) | 1152-8 (1995) | |
| gluC | Glutamate Transport | Kronemeyer et al., | X81191 |
| | Permease | Journal of | |
| | (glutamate transport | Bacteriology 177 (5): | |
| | system permease) | 1152-8 (1995) | |
| gluD | Glutamate Transport | Kronemeyer et al., | X81191 |
| | Permease | Journal of | |
| | (glutamate transport | Bacteriology 177 (5): | |
| | system permease) | 1152-8 (1995) | |
| luxR | Transcription Regulator | WO0100842 | AX065953 |
| | LuxR | EP1108790 | AX123320 |
| | (transcription regulator | | |
| | LuxR) | | |
| luxS | Histidine Kinase LuxS | EP1108790 | AX123323 |
| | (histidine kinase LuxS) | | AX127153 |
| lysR1 | Transcription Regulator | EP1108790 | AX064673 |
| | LysR1 | | AX127144 |
| | (transcription regulator | | |
| | LysR1) | | |
| lysR2 | Transcription Activator | EP1108790 | AX123312 |
| | LysR2 | | |
| | (transcription regulator | | |
| | LysR2) | | |
| lysR3 | Transcription Regulator | WO0100842 | AX065957 |
| | LysR3 | EP1108790 | AX127150 |
| | (transcription regulator | | |
| | LysR3) | | |
| menE | O-Succinylbenzoic Acid | WO0100843 | AX064599 |
| | CoA Ligase | EP1108790 | AX064193 |
| | EC 6.2.1.26 | | AX127144 |
| | (O-succinylbenzoate | | |
| | CoA ligase) | | |
| mqo | Malate-Quinone | Molenaar et al., Eur. | AJ224946 |
| | Oxidoreductase | Journal of | |
| | (malate-quinone- | Biochemistry 1; 254 | |
| | oxidoreductase) | (2): 395-403 (1998) | |
| pck | Phosphoenol Pyruvate | WO0100844 | AJ269506 |
| | Carboxykinase | | AX065053 |
| | (phosphoenol pyruvate | | |
| | carboxykinase) | | |
| pgi | Glucose 6-Phosphate | EP1087015 | AX136015 |
| | Isomerase | EP1108790 | AX127146 |
| | EC 5.3.1.9 | | |
| | (glucose-6-phosphate | | |
| | isomerase) | | |
| poxB | Pyruvate Oxidase | WO0100844 | AX064959 |
| | EC 1.2.3.3 | EP1096013 | AX137665 |
| | (pyruvate oxidase) | | |
| zwa2 | Cell Growth Factor 2 | EP1106693 | AX113822 |
| | (growth factor 2) | EP1108790 | AX127146 |

The invention accordingly also provides a process for the production of coryneform bacteria which produce L-lysine, characterized in that
a) the nucleotide sequence of a desired ORF, gene or allele of lysine production, optionally including the expression and/or regulation signals, is isolated
b) at least two copies of the nucleotide sequence of the ORF, gene or allele of lysine production are arranged in a row, preferably in tandem arrangement
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired ORF, gene or allele of lysine production at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and optionally
f) at least a third copy of the open reading frame (ORF), gene or allele of lysine production in question is introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

The invention also provides coryneform bacteria, in particular of the genus Corynebacterium, which produce L-methionine and/or L-threonine, characterized in that
a) instead of the singular copy of an open reading frame (ORF), a gene or allele of methionine production or threonine production naturally present at the particular desired site (locus), these have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
b) optionally have at least a third copy of the open reading frame (ORF), gene or allele of methionine production or threonine production mentioned at a further gene site, no nucleotide sequence which is capable of/enables episomal replication in microorganisms, no nucleotide sequence which is capable of/enables transposition and no nucleotide sequence which imparts resistance to antibiotics being present at the further gene site.

The invention also furthermore provides a process for the preparation of L-methionine and/or L-threonine, which comprises the following steps:
a) fermentation of coryneform bacteria, in particular of the genus Corynebacterium, which
   i) instead of the singular copy of an open reading frame (ORF), gene or allele of methionine production or threonine production present at the particular desired site (locus), have at least two copies of the open reading frame (ORF), gene or allele in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and
   ii) optionally have at least a third copy of the open reading frame (ORF), gene or allele of methionine production or threonine production in question at a further gene site, no nucleotide sequence which is capable of/enables episomal replication in microorganisms, no nucleotide sequence which is capable of/enables transposition and no nucleotide sequence which imparts resistance to antibiotics being present at the further gene site,
      under conditions which allow expression of the said open reading frames (ORFs), genes or alleles,
b) concentration of the L-methionine and/or L-threonine in the fermentation broth,
c) isolation of the L-methionine and/or L-threonine from the fermentation broth, optionally
d) with constituents from the fermentation broth and/or the biomass to the extent of >(greater than) 0 to 100%.

A “copy of an open reading frame (ORF), gene or allele of methionine production” is to be understood as meaning all the, preferably endogenous, open reading frames, genes or alleles of which enhancement/over-expression can have the effect of improving methionine production.

These include, inter alia, the following open reading frames, genes or alleles: accBC, accDA, aecD, cstA, cysD, cysE, cysH, cysK, cysN, cysQ, dps, eno, fda, gap, gap2, gdh, gnd, glyA, hom, hom^{FBR}, lysC, lysC^{FBR}, metA, metB, metE, metH, metY, msiK, opcA, oxyR, ppc, ppc^{FBR}, pgk, pknA, pknB, pknD, pknG, ppsA, ptsH, ptsI, ptsM, pyc, pyc P458S, sigC, sigD, sigE, sigH, sigM, tal, thyA, tkt, tpi, zwa1, zwf and zwf A213T. These are summarized and explained in Table 4. These include, in particular, the lysC^{FBR} alleles which code for a “feed back” resistant aspartate kinase (see Table 2) and the hom^{FBR} alleles which code for a “feed back” resistant homoserine dehydrogenase.

The at least third, optionally fourth or fifth copy of the open reading frame (ORF), gene or allele of methionine production in question can be integrated at a further site. The following open reading frames, genes or nucleotide sequences, inter alia, can be used for this: brnE, brnF, brnQ, ccpA1, ccpA2, citA, citB, citE, ddh, gluA, gluB, gluC, gluD, luxR, luxS, lysR1, lysR2, lysR3, menE, metD, metK, pck, pgi, poxB and zwa2. These are summarized and explained in Table 5. Intergenic regions in the chromosome, that is to say nucleotide sequences without a coding function, can furthermore be used. Finally, prophages or defective phages or DNA coding for phage components contained in the chromosome can be used for this.

TABLE 4
Open reading frames, genes and alleles of methionine
production

| | | | Access |
|---|---|---|---|
| Name | Description of the coded enzyme or protein | Reference | Number |
| accBC | Acyl-CoA Carboxylase | Jäger et al. | U35023 |
| | EC 6.3.4.14 | Archives of | |
| | (acyl-CoA carboxylase) | Microbiology | |
| | | (1996) 166: 76-82 | |
| | | EP1108790; | AX123524 |
| | | WO0100805 | AX066441 |
| accDA | Acetyl-CoA Carboxylase | EP1055725 | |
| | EC 6.4.1.2 | EP1108790 | AX121013 |
| | (acetyl-CoA carboxylase) | WO0100805 | AX066443 |
| aecD | Cystathionine beta-Lyase | Rossol et al., | M89931 |
| | EC 4.4.1.8 | Journal of | |
| | (cystathionine beta-lyase) | Bacteriology | |
| | | 174: 2968-2977 | |
| | | (1992) | |
| cstA | Carbon Starvation Protein A | EP1108790 | AX120811 |
| | (carbon starvation protein A) | WO0100804 | AX066109 |
| cysD | Sulfate Adenylyltransferase | EP1108790 | AX123177 |
| | sub-unit II | | |
| | EC 2.7.7.4 | | |
| | (sulfate adenylyltransferase small | | |
| | chain) | | |
| cysE | Serine Acetyltransferase | EP1108790 | AX122902 |
| | EC 2.3.1.30 | WO0100843 | AX063961 |
| | (serine acetyltransferase) | | |
| cysH | 3′-Phosphoadenyl Sulfate Reductase | EP1108790 | AX123178 |
| | EC 1.8.99.4 | WO0100842 | AX066001 |
| | (3′-phosphoadenosine 5′- | | |
| | phosphosulfate reductase) | | |
| cysK | Cysteine Synthase | EP1108790 | AX122901 |
| | EC 4.2.99.8 | WO0100843 | AX063963 |
| | (cysteine synthase) | | |
| cysN | Sulfate Adenylyltransferase sub- | EP1108790 | AX123176 |
| | unit I | | AX127152 |
| | EC 2.7.7.4 | | |
| | (sulfate adenylyltransferase) | | |
| cysQ | Transport protein CysQ | EP1108790 | AX127145 |
| | (transporter cysQ) | WO0100805 | AX066423 |
| dps | DNA Protection Protein | EP1108790 | AX127153 |
| | (protection during starvation | | |
| | protein) | | |
| eno | Enolase | EP1108790 | AX127146 |
| | EC 4.2.1.11 | WO0100844 | AX064945 |
| | (enolase) | EP1090998 | AX136862 |
| | | Hermann et al., | |
| | | Electrophoresis | |
| | | 19: 3217-3221 | |
| | | (1998) | |
| fda | Fructose Bisphosphate Aldolase | van der Osten et | X17313 |
| | EC 4.1.12.13 | al., Molecular | |
| | (fructose bisphosphate aldolase) | Microbiology | |
| | | 3: 1625-1637 | |
| | | (1989) | |
| gap | Glyceraldehyde 3-Phosphate | EP1108790 | AX127148 |
| | Dehydrogenase | WO0100844 | AX064941 |
| | EC 1.2.1.12 | Eikmanns et al., | X59403 |
| | (glyceraldehyde 3-phosphate | Journal of | |
| | dehydrogenase) | Bacteriology | |
| | | 174: 6076-6086 | |
| | | (1992) | |
| gap2 | Glyceraldehyde 3-Phosphate | EP1108790 | AX127146 |
| | Dehydrogenase | WO0100844 | AX064939 |
| | EC 1.2.1.12 | | |
| | (glyceraldehyde 3-phosphate | | |
| | dehydrogenase 2) | | |
| gdh | Glutamate Dehydrogenase | EP1108790 | AX127150 |
| | EC 1.4.1.4 | WO0100844 | AX063811 |
| | (glutamate dehydrogenase) | Boermann et al., | X59404 |
| | | Molecular | |
| | | Microbiology | |
| | | 6: 317-326 (1992) | |
| | | Guyonvarch et al., | X72855 |
| | | NCBI | |
| glyA | Glycine/Serine | EP1108790 | AX127146 |
| | Hydroxymethyltransferase | | AX121194 |
| | EC 2.1.2.1 | | |
| | (glycine/serine | | |
| | hydroxymethyltransferase) | | |
| gnd | 6-Phosphogluconate Dehydrogenase | EP1108790 | AX127147 |
| | EC 1.1.1.44 | | AX121689 |
| | (6-phosphogluconate dehydrogenase) | WO0100844 | AX065125 |
| hom | Homoserine Dehydrogenase | Peoples et al., | Y00546 |
| | EC 1.1.1.3 | Molecular | |
| | (homoserine dehydrogenase) | Microbiology | |
| | | 2: 63-72 (1988) | |
| hom^{FBR} | Homoserine Dehydrogenase feedback | Reinscheid et | |
| | resistant (fbr) | al., Journal of | |
| | EC 1.1.1.3 | Bacteriology | |
| | (homoserine dehydrogenase fbr) | 173: 3228-30 | |
| | | (1991) | |
| lysC | Aspartate Kinase | EP1108790 | AX120365 |
| | EC 2.7.2.4 | WO0100844 | AX063743 |
| | (aspartate kinase) | Kalinowski et | X57226 |
| | | al., Molecular | |
| | | Microbiology | |
| | | 5: 1197-204 (1991) | |
| lysC^{FBR} | Aspartate Kinase feedback | see Table 2 | |
| | resistant (fbr) | | |
| | EC 2.7.2.4 | | |
| | (aspartate kinase fbr) | | |
| metA | Homoserine Acetyltransferase | Park et al., | AF052652 |
| | EC 2.3.1.31 | Molecular Cells | |
| | (homoserine acetyltransferase) | 8: 286-94 (1998) | |
| metB | Cystathionine γ-Lyase | Hwang et al., | AF126953 |
| | EC 4.4.1.1 | Molecular Cells | |
| | (cystathionine gamma-synthase) | 9: 300-308 (1999) | |
| metE | Homocysteine Methyltransferase | EP1108790 | AX127146 |
| | EC 2.1.1.14 | | AX121345 |
| | (homocysteine methyltransferase) | | |
| metH | Homocysteine Methyltransferase | EP1108790 | AX127148 |
| | (Vitamin B12-dependent) | | AX121747 |
| | EC 2.1.1.14 | | |
| | (homocysteine methyltransferase) | | |
| metY | Acetylhomoserine Sulfhydrolase | EP1108790 | AX120810 |
| | (acetylhomoserine sulfhydrolase) | | AX127145 |
| msiK | Sugar Importer | EP1108790 | AX120892 |
| | (multiple sugar import protein) | | |
| opcA | Glucose 6-Phosphate Dehydrogenase | WO0104325 | AX076272 |
| | (subunit of glucose 6-phosphate | | |
| | dehydrogenase) | | |
| oxyR | Transcription Regulator | EP1108790 | AX122198 |
| | (transcriptional regulator) | | AX127149 |
| ppc^{FBR} | Phosphoenol Pyruvate Carboxylase | EP0723011 | |
| | feedback resistent | WO0100852 | |
| | EC 4.1.1.31 | | |
| | (phosphoenol pyruvate carboxylase | | |
| | feedback resistant) | | |
| ppc | Phosphoenol Pyruvate Carboxylase | EP1108790 | AX127148 |
| | EC 4.1.1.31 | | AX123554 |
| | (phosphoenol pyruvate carboxylase) | O'Reagan et al., | M25819 |
| | | Gene 77(2): 237-251 | |
| | | (1989) | |
| pgk | Phosphoglycerate Kinase | EP1108790 | AX121838 |
| | EC 2.7.2.3 | | AX127148 |
| | (phosphoglycerate kinase) | WO0100844 | AX064943 |
| | | Eikmanns, Journal | X59403 |
| | | of Bacteriology | |
| | | 174: 6076-6086 | |
| | | (1992) | |
| pknA | Protein Kinase A | EP1108790 | AX120131 |
| | (protein kinase A) | | AX120085 |
| pknB | Protein Kinase B | EP1108790 | AX120130 |
| | (protein kinase B) | | AX120085 |
| pknD | Protein Kinase D | EP1108790 | AX127150 |
| | (protein kinase D) | | AX122469 |
| | | | AX122468 |
| pknG | Protein Kinase G | EP1108790 | AX127152 |
| | (protein kinase G) | | AX123109 |
| ppsA | Phosphoenol Pyruvate Synthase | EP1108790 | AX127144 |
| | EC 2.7.9.2 | | AX120700 |
| | (phosphoenol pyruvate synthase) | | AX122469 |
| ptsH | Phosphotransferase System Protein H | EP1108790 | AX122210 |
| | | | AX127149 |
| | EC 2.7.1.69 | WO0100844 | AX069154 |
| | (phosphotransferase system | | |
| | component H) | | |
| ptsI | Phosphotransferase System Enzyme I | EP1108790 | AX122206 |
| | EC 2.7.3.9 | | AX127149 |
| | (phosphotransferase system enzyme | | |
| | I) | | |
| ptsM | Glucose-specific | Lee et al., FEMS | L18874 |
| | Phosphotransferase System Enzyme | Microbiology | |
| | II | Letters 119(1-2): | |
| | EC 2.7.1.69 | 137-145 (1994) | |
| | (glucose phosphotransferase-system | | |
| | enzyme II) | | |
| pyc | Pyruvate Carboxylase | WO9918228 | A97276 |
| | EC 6.4.1.1 | Peters-Wendisch | Y09548 |
| | (pyruvate carboxylase) | et al., | |
| | | Microbiology | |
| | | 144: 915-927 | |
| | | (1998) | |
| pyc | Pyruvate Carboxylase | EP1108790 | |
| P458S | EC 6.4.1.1 | | |
| | (pyruvate carboxylase) | | |
| | amino acid exchange P458S | | |
| sigC | Sigma Factor C | EP1108790 | AX120368 |
| | EC 2.7.7.6 | | AX120085 |
| | (extracytoplasmic function | | |
| | alternative sigma factor C) | | |
| sigD | RNA Polymerase Sigma Factor D | EP1108790 | AX120753 |
| | EC 2.7.7.6 | | AX127144 |
| | (RNA polymerase sigma factor) | | |
| sigE | Sigma Factor E | EP1108790 | AX127146 |
| | EC 2.7.7.6 | | AX121325 |
| | (extracytoplasmic function | | |
| | alternative sigma factor E) | | |
| sigH | Sigma Factor H | EP1108790 | AX127145 |
| | EC 2.7.7.6 | | AX120939 |
| | (sigma factor SigH) | | |
| sigM | Sigma Factor M | EP1108790 | AX123500 |
| | EC 2.7.7.6 | | AX127153 |
| | (sigma factor SigM) | | |
| tal | Transaldolase | WO0104325 | AX076272 |
| | EC 2.2.1.2 | | |
| | (transaldolase) | | |
| thyA | Thymidylate Synthase | EP1108790 | AX121026 |
| | EC 2.1.1.45 | | AX127145 |
| | (thymidylate synthase) | | |
| tkt | Transketolase | Ikeda et al., | AB023377 |
| | EC 2.2.1.1 | NCBI | |
| | (transketolase) | | |
| tpi | Triose Phosphate Isomerase | Eikmanns, Journal | X59403 |
| | EC 5.3.1.1 | of Bacteriology | |
| | (triose phosphate isomerase) | 174: 6076-6086 | |
| | | (1992) | |
| zwa1 | Cell Growth Factor 1 | EP1111062 | AX133781 |
| | (growth factor 1) | | |
| zwf | Glucose 6-Phosphate 1- | EP1108790 | AX127148 |
| | Dehydrogenase | | AX121827 |
| | EC 1.1.1.49 | WO0104325 | AX076272 |
| | (glucose 6-phosphate 1- | | |
| | dehydrogenase) | | |
| zwf | Glucose 6-Phosphate 1- | EP1108790 | |
| A213T | Dehydrogenase | | |
| | EC 1.1.1.49 | | |
| | (glucose 6-phosphate 1- | | |
| | dehydrogenase) | | |
| | amino acid exchange A213T | | |

TABLE 5
Further gene sites for integration of open reading frames,
genes and alleles of methionine production

| | Description | | |
|---|---|---|---|
| Gene name | of the coded enzyme or protein | Reference | Access Number |
| brnE | Transporter of | EP1096010 | AX137709 |
| | branched-chain amino | | AX137714 |
| | acids | | |
| | (branched-chain amino | | |
| | acid transporter) | | |
| brnF | Transporter of | EP1096010 | AX137709 |
| | branched-chain amino | | AX137714 |
| | acids | | |
| | (branched-chain amino | | |
| | acid transporter) | | |
| brnQ | Carrier protein of | Tauch et al., Archives | M89931 |
| | branched-chain amino | of Microbiology | AX066841 |
| | acids | 169(4): 303-12 (1998) | AX127150 |
| | (branched-chain amino | WO0100805 | |
| | acid transport system | EP1108790 | |
| | carrier protein) | | |
| ccpA1 | Catabolite Control | WO0100844 | AX065267 |
| | Protein | EP1108790 | AX127147 |
| | (catabolite control | | |
| | protein A1) | | |
| ccpA2 | Catabolite Control | WO0100844 | AX065267 |
| | Protein | EP1108790 | AX121594 |
| | (catabolite control | | |
| | protein A2) | | |
| citA | Sensor Kinase CitA | EP1108790 | AX120161 |
| | (sensor kinase CitA) | | |
| citB | Transcription Regulator | EP1108790 | AX120163 |
| | CitB | | |
| | (transcription | | |
| | regulator CitB) | | |
| citE | Citrate Lyase | WO0100844 | AX065421 |
| | EC 4.1.3.6 | EP1108790 | AX127146 |
| | (citrate lyase) | | |
| ddh | Diaminopimelate | Ishino et al., Nucleic | S07384 |
| | Dehydrogenase | Acids Research 15: 3917 | AX127152 |
| | EC 1.4.1.16 | (1987) | |
| | (diaminopimelate | EP1108790 | |
| | dehydrogenase) | | |
| gluA | Glutamate Transport | Kronemeyer et al., | X81191 |
| | ATP-binding Protein | Journal of Bacteriology | |
| | (glutamate transport | 177(5): 1152-8 (1995) | |
| | ATP-binding protein) | | |
| gluB | Glutamate-binding | Kronemeyer et al., | X81191 |
| | Protein | Journal of Bacteriology | |
| | (glutamate-binding | 177(5): 1152-8 (1995) | |
| | protein) | | |
| gluC | Glutamate Transport | Kronemeyer et al., | X81191 |
| | Permease | Journal of Bacteriology | |
| | (glutamate transport | 177(5): 1152-8 (1995) | |
| | system permease) | | |
| gluD | Glutamate Transport | Kronemeyer et al., | X81191 |
| | Permease | Journal of Bacteriology | |
| | (glutamate transport | 177(5): 1152-8 (1995) | |
| | system permease) | | |
| luxR | Transcription Regulator | WO0100842 | AX065953 |
| | LuxR | EP1108790 | AX123320 |
| | (transcription | | |
| | regulator LuxR) | | |
| luxS | Histidine Kinase LuxS | EP1108790 | AX123323 |
| | (histidine kinase LuxS) | | AX127153 |
| lysR1 | Transcription Regulator | EP1108790 | AX064673 |
| | LysR1 | | AX127144 |
| | (transcription | | |
| | regulator LysR1) | | |
| lysR2 | Transcription Activator | EP1108790 | AX123312 |
| | LysR2 | | |
| | (transcription | | |
| | regulator LysR2) | | |
| lysR3 | Transcription Regulator | WO0100842 | AX065957 |
| | LysR3 | EP1108790 | AX127150 |
| | (transcription | | |
| | regulator LysR3) | | |
| menE | O-Succinylbenzoic Acid | WO0100843 | AX064599 |
| | CoA Ligase | EP1108790 | AX064193 |
| | EC 6.2.1.26 | | AX127144 |
| | (O-succinylbenzoate CoA | | |
| | ligase) | | |
| metD | Transcription Regulator | EP1108790 | AX123327 |
| | MetD | | AX127153 |
| | (transcription | | |
| | regulator MetD) | | |
| metK | Methionine Adenosyl | WO0100843 | AX063959 |
| | Transferase | EP1108790 | AX127148 |
| | EC 2.5.1.6 | | |
| | (S-adenosylmethionine | | |
| | synthetase) | | |
| pck | Phosphoenol Pyruvate | WO0100844 | AJ269506 |
| | Carboxykinase | | AX065053 |
| | (phosphoenol pyruvate | | |
| | carboxykinase) | | |
| pgi | Glucose 6-Phosphate | EP1087015 | AX136015 |
| | Isomerase | EP1108790 | AX127146 |
| | EC 5.3.1.9 | | |
| | (glucose-6-phosphate | | |
| | isomerase) | | |
| poxB | Pyruvate Oxidase | WO0100844 | AX064959 |
| | EC 1.2.3.3 | EP1096013 | AX137665 |
| | (pyruvate oxidase) | | |
| zwa2 | Cell Growth Factor 2 | EP1106693 | AX113822 |
| | (growth factor 2) | EP1108790 | AX127146 |

A “copy of an open reading frame (ORF), gene or allele of threonine production” is to be understood as meaning all the, preferably endogenous, open reading frames, genes or alleles of which enhancement/over-expression can have the effect of improving threonine production.

These include, inter alia, the following open reading frames, genes or alleles: accBC, accDA, cstA, cysD, cysE, cysH, cysI, cysN, cysQ, dps, eno, fda, gap, gap2, gdh, gnd, hom, hom^{FBR}, lysC, lysC^{FBR}, msiK, opcA, oxyR, ppc, ppc^{FBR}, pgk, pknA, pknB, pknD, pknG, ppsA, ptsH, ptsI, ptsM, pyc, pyc P458S, sigC, sigD, sigE, sigH, sigM, tal, thyA, tkt, tpi, thrB, thrC, thrE, zwa1, zwf and zwf A213T. These are summarized and explained in Table 6. These include, in particular, the lysC^{FBR} alleles which code for a “feed back” resistant aspartate kinase (See Table 2) and the hom^{FBR} alleles which code for a “feed back” resistant homoserine dehydrogenase.

The at least third, optionally fourth or fifth copy of the open reading frame (ORF), gene or allele of threonine production in question can be integrated at a further site. The following open reading frames, genes or nucleotide sequences, inter alia, can be used for this: ccpA1, ccpA2, citA, citB, citE, ddh, gluA, gluB, gluC, gluD, glyA, ilvA, ilvBN, ilvC, ilvD, luxR, luxS, lysR1, lysR2, lysR3, mdh, menE, metA, metD, pck, poxB, sigB and zwa2. These are summarized and explained in Table 7. Intergenic regions in the chromosome, that is to say nucleotide sequences without a coding function, can furthermore be used. Finally, prophages or defective phages or DNA coding for phage components contained in the chromosome can be used for this.

TABLE 6
Open reading frames, genes and alleles of threonine
production

| | Description of the coded enzyme or | | Access |
|---|---|---|---|
| Name | protein | Reference | Number |
| accBC | Acyl-CoA Carboxylase | Jäger et al. | U35023 |
| | EC 6.3.4.14 | Archives of | |
| | (acyl-CoA carboxylase) | Microbiology | |
| | | (1996) 166: 76-82 | |
| | | EP1108790; | AX123524 |
| | | WO0100805 | AX066441 |
| accDA | Acetyl-CoA Carboxylase | EP1055725 | |
| | EC 6.4.1.2 | EP1108790 | AX121013 |
| | (acetyl-CoA carboxylase) | WO0100805 | AX066443 |
| cstA | Carbon Starvation Protein A | EP1108790 | AX120811 |
| | (carbon starvation protein A) | WO0100804 | AX066109 |
| cysD | Sulfate Adenylyltransferase | EP1108790 | AX123177 |
| | sub-unit II | | |
| | EC 2.7.7.4 | | |
| | (sulfate adenylyltransferase small | | |
| | chain) | | |
| cysE | Serine Acetyltransferase | EP1108790 | AX122902 |
| | EC 2.3.1.30 | WO0100843 | AX063961 |
| | (serine acetyltransferase) | | |
| cysH | 3′-Phosphoadenyl Sulfate Reductase | EP1108790 | AX123178 |
| | EC 1.8.99.4 | WO0100842 | AX066001 |
| | (3′-phosphoadenosine 5′-phosphosulfate | | |
| | reductase) | | |
| cysK | Cysteine Synthase | EP1108790 | AX122901 |
| | EC 4.2.99.8 | WO0100843 | AX063963 |
| | (cysteine synthase) | | |
| cysN | Sulfate Adenylyltransferase sub-unit I | EP1108790 | AX123176 |
| | EC 2.7.7.4 | | AX127152 |
| | (sulfate adenylyltransferase) | | |
| cysQ | Transport protein CysQ | EP1108790 | AX127145 |
| | (transporter cysQ) | WO0100805 | AX066423 |
| dps | DNA Protection Protein | EP1108790 | AX127153 |
| | (protection during starvation protein) | | |
| eno | Enolase | EP1108790 | AX127146 |
| | EC 4.2.1.11 | WO0100844 | AX064945 |
| | (enolase) | EP1090998 | AX136862 |
| | | Hermann et al., | |
| | | Electrophoresis | |
| | | 19: 3217-3221 | |
| | | (1998) | |
| fda | Fructose Bisphosphate Aldolase | van der Osten | X17313 |
| | EC 4.1.2.13 | et al., | |
| | (fructose bisphosphate aldolase) | Molecular | |
| | | Microbiology | |
| | | 3: 1625-1637 | |
| | | (1989) | |
| gap | Glyceraldehyde 3-Phosphate Dehydrogenase | EP1108790 | AX127148 |
| | EC 1.2.1.12 | WO0100844 | AX064941 |
| | (glyceraldehyde 3-phosphate | Eikmanns et | X59403 |
| | dehydrogenase) | al., Journal of | |
| | | Bacteriology | |
| | | 174: 6076-6086 | |
| | | (1992) | |
| gap2 | Glyceraldehyde 3-Phosphate Dehydrogenase | EP1108790 | AX127146 |
| | EC 1.2.1.12 | WO0100844 | AX064939 |
| | (glyceraldehyde 3-phosphate | | |
| | dehydrogenase 2) | | |
| gdh | Glutamate Dehydrogenase | EP1108790 | AX127150 |
| | EC 1.4.1.4 | WO0100844 | AX063811 |
| | (glutamate dehydrogenase) | Boermann et | X59404 |
| | | al., Molecular | |
| | | Microbiology | |
| | | 6: 317-326 | |
| | | (1992) | |
| | | Guyonvarch et | X72855 |
| | | al, NCBI | |
| gnd | 6-Phosphogluconate Dehydrogenase | EP1108790 | AX127147 |
| | EC 1.1.1.44 | | AX121689 |
| | (6-phosphogluconate dehydrogenase) | WO0100844 | AX065125 |
| hom | Homoserine Dehydrogenase | Peoples et al., | Y00546 |
| | EC 1.1.1.3 | Molecular | |
| | (homoserine dehydrogenase) | Microbiology | |
| | | 2: 63-72 (1988) | |
| hom^{FBR} | Homoserine Dehydrogenase feedback | Reinscheid et | |
| | resistant (fbr) | al., Journal of | |
| | EC 1.1.1.3 | Bacteriology | |
| | (homoserine dehydrogenase fbr) | 173: 3228-30 | |
| | | (1991) | |
| lysC | Aspartate Kinase | EP1108790 | AX120365 |
| | EC 2.7.2.4 | WO0100844 | AX063743 |
| | (aspartate kinase) | Kalinowski et | X57226 |
| | | al., Molecular | |
| | | Microbiology | |
| | | 5: 1197-204 | |
| | | (1991) | |
| lysC^{FBR} | Aspartate Kinase feedback resistent | see Table 2 | |
| | (fbr) | | |
| | EC 2.7.2.4 | | |
| | (aspartate kinase fbr) | | |
| msiK | Sugar Importer | EP1108790 | AX120892 |
| | (multiple sugar import protein) | | |
| opcA | Glucose 6-Phosphate Dehydrogenase | WO0104325 | AX076272 |
| | (subunit of glucose 6-phosphate | | |
| | dehydrogenase) | | |
| oxyR | Transcription Regulator | EP1108790 | AX122198 |
| | (transcriptional regulator) | | AX127149 |
| ppc^{FBR} | Phosphoenol Pyruvate Carboxylase | EP0723011 | |
| | feedback resistent | WO0100852 | |
| | EC 4.1.1.31 | | |
| | (phosphoenol pyruvate carboxylase | | |
| | feedback resistant) | | |
| ppc | Phosphoenol Pyruvate Carboxylase | EP1108790 | AX127148 |
| | EC 4.1.1.31 | | AX123554 |
| | (phosphoenol pyruvate carboxylase) | O'Reagan et | M25819 |
| | | al., Gene | |
| | | 77(2): 237-251 | |
| | | (1989) | |
| pgk | Phosphoglycerate Kinase | EP1108790 | AX121838 |
| | EC 2.7.2.3 | | AX127148 |
| | (phosphoglycerate kinase) | WO0100844 | AX064943 |
| | | Eikmanns, | X59403 |
| | | Journal of | |
| | | Bacteriology | |
| | | 174: 6076-6086 | |
| | | (1992) | |
| pknA | Protein Kinase A | EP1108790 | AX120131 |
| | (protein kinase A) | | AX120085 |
| pknB | Protein Kinase B | EP1108790 | AX120130 |
| | (protein kinase B) | | AX120085 |
| pknD | Protein Kinase D | EP1108790 | AX127150 |
| | (protein kinase D) | | AX122469 |
| | | | AX122468 |
| pknG | Protein Kinase G | EP1108790 | AX127152 |
| | (protein kinase G) | | AX123109 |
| ppsA | Phosphoenol Pyruvate Synthase | EP1108790 | AX127144 |
| | EC 2.7.9.2 | | AX120700 |
| | (phosphoenol pyruvate synthase) | | AX122469 |
| ptsH | Phosphotransferase System Protein H | EP1108790 | AX122210 |
| | EC 2.7.1.69 | | AX127149 |
| | (phosphotransferase system component H) | WO0100844 | AX069154 |
| ptsI | Phosphotransferase System Enzyme I | EP1108790 | AX122206 |
| | EC 2.7.3.9 | | AX127149 |
| | (phosphotransferase system enzyme I) | | |
| ptsM | Glucose-specific Phosphotransferase | Lee et al., | L18874 |
| | System Enzyme II | FEMS | |
| | EC 2.7.1.69 | Microbiology | |
| | (glucose phosphotransferase-system | Letters 119(1-2): | |
| | enzyme II) | 137-145 | |
| | | (1994) | |
| pyc | Pyruvate Carboxylase | WO9918228 | A97276 |
| | EC 6.4.1.1 | Peters-Wendisch | Y09548 |
| | (pyruvate carboxylase) | et al., | |
| | | Microbiology | |
| | | 144: 915-927 | |
| | | (1998) | |
| pyc | Pyruvate Carboxylase | EP1108790 | |
| P458S | EC 6.4.1.1 | | |
| | (pyruvate carboxylase) | | |
| | amino acid exchange P458S | | |
| sigC | Sigma Factor C | EP1108790 | AX120368 |
| | EC 2.7.7.6 | | AX120085 |
| | (extracytoplasmic function alternative | | |
| | sigma factor C) | | |
| sigD | RNA Polymerase Sigma Factor D | EP1108790 | AX120753 |
| | EC 2.7.7.6 | | AX127144 |
| | (RNA polymerase sigma factor) | | |
| sigE | Sigma Factor E | EP1108790 | AX127146 |
| | EC 2.7.7.6 | | AX121325 |
| | (extracytoplasmic function alternative | | |
| | sigma factor E) | | |
| sigH | Sigma Factor H | EP1108790 | AX127145 |
| | EC 2.7.7.6 | | AX120939 |
| | (sigma factor SigH) | | |
| sigM | Sigma Factor M | EP1108790 | AX123500 |
| | EC 2.7.7.6 | | AX127153 |
| | (sigma factor SigM) | | |
| tal | Transaldolase | WO0104325 | AX076272 |
| | EC 2.2.1.2 | | |
| | (transaldolase) | | |
| thrB | Homoserine Kinase | Peoples et al., | Y00546 |
| | EC 2.7.1.39 | Molecular | |
| | (homoserine kinase) | Microbiology | |
| | | 2: 63-72 (1988) | |
| thrC | Threonine Synthase | Han et al., | X56037 |
| | EC 4.2.99.2 | Molecular | |
| | (threonine synthase) | Microbiology | |
| | | 4: 1693-1702 | |
| | | (1990) | |
| thrE | Threonine Exporter | EP1085091 | AX137526 |
| | (threonine export carrier) | | |
| thyA | Thymidylate Synthase | EP1108790 | AX121026 |
| | EC 2.1.1.45 | | AX127145 |
| | (thymidylate synthase) | | |
| tkt | Transketolase | Ikeda et al., | AB023377 |
| | EC 2.2.1.1 | NCBI | |
| | (transketolase) | | |
| tpi | Triose Phosphate Isomerase | Eikmanns, | X59403 |
| | EC 5.3.1.1 | Journal of | |
| | (triose phosphate isomerase) | Bacteriology | |
| | | 174: 6076-6086 | |
| | | (1992) | |
| zwal | Cell Growth Factor 1 | EP1111062 | AX133781 |
| | (growth factor 1) | | |
| zwf | Glucose 6-Phosphate 1-Dehydrogenase | EP1108790 | |
| | EC 1.1.1.49 | | |
| | (glucose 6-phosphate 1-dehydrogenase) | WO0104325 | |
| zwf | Glucose 6-Phosphate 1-Dehydrogenase | EP1108790 | AX127148 |
| A213T | EC 1.1.1.49 | | AX121827 |
| | (glucose 6-phosphate 1-dehydrogenase) | | AX076272 |
| | amino acid exchange A213T | | |

TABLE 7
Further gene sites for integration of open reading frames,
genes and alleles of threonine production

| | Description of the coded | | |
|---|---|---|---|
| Gene name | enzyme or protein | Reference | Access Number |
| ccpA1 | Catabolite Control | WO0100844 | AX065267 |
| | Protein | EP1108790 | AX127147 |
| | (catabolite control | | |
| | protein A1) | | |
| ccpA2 | Catabolite Control | WO0100844 | AX065267 |
| | Protein | EP1108790 | AX121594 |
| | (catabolite control | | |
| | protein A2) | | |
| citA | Sensor Kinase CitA | EP1108790 | AX120161 |
| | (sensor kinase CitA) | | |
| citB | Transcription Regulator | EP1108790 | AX120163 |
| | CitB | | |
| | (transcription regulator | | |
| | CitB) | | |
| citE | Citrate Lyase | WO0100844 | AX065421 |
| | EC 4.1.3.6 | EP1108790 | AX127146 |
| | (citrate lyase) | | |
| ddh | Diaminopimelate | Ishino et al., Nucleic | S07384 |
| | Dehydrogenase | Acids Research 15: 3917 | AX127152 |
| | EC 1.4.1.16 | (1987) | |
| | (diaminopimelate | EP1108790 | |
| | dehydrogenase) | | |
| gluA | Glutamate Transport ATP- | Kronemeyer et al., | X81191 |
| | binding Protein | Journal of Bacteriology | |
| | (glutamate transport ATP- | 177(5): 1152-8 (1995) | |
| | binding protein) | | |
| gluB | Glutamate-binding Protein | Kronemeyer et al., | X81191 |
| | (glutamate-binding | Journal of Bacteriology | |
| | protein) | 177(5): 1152-8 (1995) | |
| gluC | Glutamate Transport | Kronemeyer et al., | X81191 |
| | Permease | Journal of Bacteriology | |
| | (glutamate transport | 177(5): 1152-8 (1995) | |
| | system permease) | | |
| gluD | Glutamate Transport | Kronemeyer et al., | X81191 |
| | Permease | Journal of Bacteriology | |
| | (glutamate transport | 177(5): 1152-8 (1995) | |
| | system permease) | | |
| glyA | Glycine | WO0100843 | AX063861 |
| | Hydroxymethyltransferase | | AF327063 |
| | EC 2.1.2.1 | | |
| | (glycine | | |
| | hydroxymethyltransferase) | | |
| ilvA | Threonine Dehydratase | Möckel et al., Journal | A47044 |
| | EC 4.2.1.16 | of Bacteriology 174 | L01508 |
| | (threonine dehydratase) | (24), 8065-8072 (1992) | AX127150 |
| | | EP1108790 | |
| ilvBN | Acetolactate Synthase | Keilhauer et al., | A48648 |
| | EC 4.1.3.18 | Journal of Bacteriology | L09232 |
| | (acetolactate synthase) | 175(17): 5595-603 (1993) | AX127147 |
| | | EP1108790 | |
| ilvC | Reductoisomerase | Keilhauer et al., | C48648 |
| | EC 1.1.1.86 | Journal of Bacteriology | AX127147 |
| | (ketol-acid | 175(17): 5595-603 (1993) | |
| | reductoisomerase) | EP1108790 | |
| ilvD | Dihydroxy-acid | EP1006189 | AX136925 |
| | Dehydratase | | |
| | EC 4.2.1.9 | | |
| | (dihydroxy-acid | | |
| | dehydratase) | | |
| luxR | Transcription Regulator | WO0100842 | AX065953 |
| | LuxR | EP1108790 | AX123320 |
| | (transcription regulator | | |
| | LuxR) | | |
| luxS | Histidine Kinase LuxS | EP1108790 | AX123323 |
| | (histidine kinase LuxS) | | AX127153 |
| lysR1 | Transcription Regulator | EP1108790 | AX064673 |
| | LysR1 | | AX127144 |
| | (transcription regulator | | |
| | LysR1) | | |
| lysR2 | Transcription Activator | EP1108790 | AX123312 |
| | LysR2 | | |
| | (transcription regulator | | |
| | LysR2) | | |
| lysR3 | Transcription Regulator | WO0100842 | AX065957 |
| | LysR3 | EP1108790 | AX127150 |
| | (transcription regulator | | |
| | LysR3) | | |
| mdh | Malate Dehydrogenase | WO0100844 | AX064895 |
| | EC 1.1.1.37 | | |
| | (malate dehydrogenase) | | |
| menE | O-Succinylbenzoic Acid | WO0100843 | AX064599 |
| | CoA Ligase | EP1108790 | AX064193 |
| | EC 6.2.1.26 | | AX127144 |
| | (O-succinylbenzoate CoA | | |
| | ligase) | | |
| metA | Homoserine O- | Park et al., Molecular | AX063895 |
| | Acetyltransferase | Cells 30; 8(3): 286-94 | AX127145 |
| | EC 2.3.1.31 | (1998) | |
| | (homoserine O- | WO0100843 | |
| | acetyltransferase) | EP1108790 | |
| metD | Transcription Regulator | EP1108790 | AX123327 |
| | MetD | | AX127153 |
| | (transcription regulator | | |
| | MetD) | | |
| pck | Phosphoenol Pyruvate | WO0100844 | AJ269506 |
| | Carboxykinase | | AX065053 |
| | (phosphoenol pyruvate | | |
| | carboxykinase) | | |
| poxB | Pyruvate Oxidase | WO0100844 | AX064959 |
| | EC 1.2.3.3 | EP1096013 | AX137665 |
| | (pyruvate oxidase) | | |
| sigB | RNA Polymerase | EP1108790 | AX127149 |
| | Transcription Factor | | |
| | (RNA polymerase | | |
| | transcription factor) | | |
| zwa2 | Cell Growth Factor 2 | EP1106693 | AX113822 |
| | (growth factor 2) | EP1108790 | AX127146 |

The invention accordingly also provides a process for the production of coryneform bacteria which produce L-methionine and/or L-threonine, characterized in that
a) the nucleotide sequence of a desired ORF, gene or allele of methionine production or threonine production, optionally including the expression and/or regulation signals, is isolated
b) at least two copies of the nucleotide sequence of the ORF, gene or allele of methionine production or threonine production are arranged in a row, preferably in tandem arrangement
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired ORF, gene or allele of methionine or threonine production at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and optionally
f) at least a third copy of the open reading frame (ORF), gene or allele of methionine production or threonine production in question is introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

The invention also provides coryneform bacteria, in particular of the genus Corynebacterium, which produce L-valine, characterized in that
a) instead of the singular copy of an open reading frame (ORF), a gene or allele of valine production naturally present at the particular desired site (locus), these have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
b) optionally have at least a third copy of the open reading frame (ORF), gene or allele of valine production mentioned at a further gene site, no nucleotide sequence which is capable of/enables episomal replication in microorganisms, no nucleotide sequence which is capable of/enables transposition and no nucleotide sequence which imparts resistance to antibiotics being present at the further gene site.

The invention also furthermore provides a process for the preparation of L-valine, which comprises the following steps:
a) fermentation of coryneform bacteria, in particular of the genus Corynebacterium, which
   i) instead of the singular copy of an open reading frame (ORF), gene or allele of valine production present at the particular desired site (locus), have at least two copies of the open reading frame (ORF), gene or allele in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and
   ii) optionally have at least a third copy of the open reading frame (ORF), gene or allele of valine production in question at a further gene site, no nucleotide sequence which is capable of/enables episomal replication in microorganisms, no nucleotide sequence which is capable of/enables transposition and no nucleotide sequence which imparts resistance to antibiotics being present at the further gene site,
      under conditions which allow expression of the said open reading frames (ORFs), genes or alleles,
b) concentration of the L-valine in the fermentation broth,
c) isolation of the L-valine from the fermentation broth, optionally
d) with constituents from the fermentation broth and/or the biomass to the extent of >(greater than) 0 to 100%.

A “copy of an open reading frame (ORF), gene or allele of valine production” is to be understood as meaning all the, preferably endogenous, open reading frames, genes or alleles of which enhancement/over-expression can have the effect of improving valine production.

These include, inter alia, the following open reading frames, genes or alleles: brnE, brnF, brnEF, cstA, cysD, dps, eno, fda, gap, gap2, gdh, ilvB, ilvN, ilvBN, ilvC, ilvD, ilvE msiK, pgk, ptsH, ptsI, ptsM, sigC, sigD, sigE, sigH, sigM, tpi and zwa1. These are summarized and explained in Table 8. These include in particular the ilvBN alleles which code for a valine-resistant acetolactate synthase.

The at least third, optionally fourth or fifth copy of the open reading frame (ORF), gene or allele of valine production in question can be integrated at a further site. The following open reading frames, genes or nucleotide sequences, inter alia, can be used for this: aecD, ccpA1, ccpA2, citA, citB, citE, ddh, gluA, gluB, gluC, gluD, glyA, ilvA, luxR, lysR1, lysR2, lysR3, panB, panC, poxB and zwa2. These are summarized and explained in Table 9. Intergenic regions in the chromosome, that is to say nucleotide sequences without a coding function, can furthermore be used. Finally, prophages or defective phages or DNA coding for phage components contained in the chromosome can be used for this.

TABLE 8
Open reading frames, genes and alleles of valine production

| Name | Description of the coded enzyme or protein | Reference | Access Number |
|---|---|---|---|
| brnEF | Export of branched-chain amino | EP1096010 | AF454053 |
| | acids | Kennerknecht et | |
| | (branched chain amino acid export) | al., NCBI | |
| cstA | Carbon Starvation Protein A | EP1108790 | AX120811 |
| | (carbon starvation protein A) | WO0100804 | AX066109 |
| dps | DNA Protection Protein | EP1108790 | AX127153 |
| | (protection during starvation | | |
| | protein) | | |
| eno | Enolase | EP1108790 | AX127146 |
| | EC 4.2.1.11 | WO0100844 | AX064945 |
| | (enolase) | EP1090998 | AX136862 |
| | | Hermann et al., | |
| | | Electrophoresis | |
| | | 19: 3217-3221 | |
| | | (1998) | |
| fda | Fructose Bisphosphate Aldolase | van der Osten et | X17313 |
| | EC 4.1.2.13 | al., Molecular | |
| | (fructose bisphosphate aldolase) | Microbiology | |
| | | 3: 1625-1637 | |
| | | (1989) | |
| gap | Glyceraldehyde 3-Phosphate | EP1108790 | AX127148 |
| | Dehydrogenase | WO0100844 | AX064941 |
| | EC 1.2.1.12 | Eikmanns et al., | X59403 |
| | (glyceraldehyde 3-phosphate | Journal of | |
| | dehydrogenase) | Bacteriology | |
| | | 174: 6076-6086 | |
| | | (1992) | |
| gap2 | Glyceraldehyde 3-Phosphate | EP1108790 | AX127146 |
| | Dehydrogenase | WO0100844 | AX064939 |
| | EC 1.2.1.12 | | |
| | (glyceraldehyde 3-phosphate | | |
| | dehydrogenase 2) | | |
| gdh | Glutamate Dehydrogenase | EP1108790 | AX127150 |
| | EC 1.4.1.4 | WO0100844 | AX063811 |
| | (glutamate dehydrogenase) | Boermann et al., | X59404 |
| | | Molecular | |
| | | Microbiology | |
| | | 6: 317-326 | |
| | | (1992); | |
| | | Guyonvarch et | X72855 |
| | | al., NCBI | |
| ilvBN | Acetolactate Synthase | Keilhauer et | L09232 |
| | EC 4.1.3.18 | al., Journal of | |
| | (acetolactate synthase) | Bacteriology | |
| | | 175(17): 5595-603 | |
| | | (1993) | |
| | | EP1108790 | AX127147 |
| ilvC | Isomeroreductase | Keilhauer et | C48648 |
| | EC 1.1.1.86 | al., Journal of | AX127147 |
| | (acetohydroxy acid | Bacteriology | |
| | isomeroreductase) | 175(17): 5595-603 | |
| | | (1993) | |
| | | EP1108790 | |
| ilvD | Dihydroxy-acid Dehydratase | EP1006189 | AX136925 |
| | EC 4.2.1.9 | | |
| | (dihydroxy acid dehydratase) | | |
| ilvE | Transaminase B | EP1108790 | AX127150 |
| | EC 2.6.1.42 | | AX122498 |
| | (transaminase B) | | |
| msiK | Sugar Importer | EP1108790 | AX120892 |
| | (multiple sugar import protein) | | |
| pgk | Phosphoglycerate Kinase | EP1108790 | AX121838 |
| | EC 2.7.2.3 | | AX127148 |
| | (phosphoglycerate kinase) | WO0100844 | AX064943 |
| | | Eikmanns, | X59403 |
| | | Journal of | |
| | | Bacteriology | |
| | | 174: 6076-6086 | |
| | | (1992) | |
| ptsH | Phosphotransferase System Protein H | EP1108790 | AX122210 |
| | EC 2.7.1.69 | | AX127149 |
| | (phosphotransferase system | WO0100844 | AX069154 |
| | component H) | | |
| ptsI | Phosphotransferase System Enzyme I | EP1108790 | AX122206 |
| | EC 2.7.3.9 | | AX127149 |
| | (phosphotransferase system enzyme | | |
| | I) | | |
| ptsM | Glucose-specific Phosphotransferase | Lee et al., FEMS | L18874 |
| | System Enzyme II | Microbiology | |
| | EC 2.7.1.69 | Letters 119(1-2): | |
| | (glucose phosphotransferase-system | 137-145 | |
| | enzyme II) | (1994) | |
| sigC | Sigma Factor C | EP1108790 | AX120368 |
| | EC 2.7.7.6 | | AX120085 |
| | (extracytoplasmic function | | |
| | alternative sigma factor C) | | |
| sigD | RNA Polymerase Sigma Factor D | EP1108790 | AX120753 |
| | EC 2.7.7.6 | | AX127144 |
| | (RNA polymerase sigma factor) | | |
| sigE | Sigma Factor E | EP1108790 | AX127146 |
| | EC 2.7.7.6 | | AX121325 |
| | (extracytoplasmic function | | |
| | alternative sigma factor E) | | |
| sigH | Sigma Factor H | EP1108790 | AX127145 |
| | EC 2.7.7.6 | | AX120939 |
| | (sigma factor SigH) | | |
| sigM | Sigma Factor M | EP1108790 | AX123500 |
| | EC 2.7.7.6 | | AX127153 |
| | (sigma factor SigM) | | |
| tpi | Triose Phosphate Isomerase | Eikmanns, | X59403 |
| | EC 5.3.1.1 | Journal of | |
| | (triose phosphate isomerase) | Bacteriology | |
| | | 174: 6076-6086 | |
| | | (1992) | |
| zwa1 | Cell Growth Factor 1 | EP1111062 | AX133781 |
| | (growth factor 1) | | |

TABLE 9
Further gene sites for integration of open reading frames,
genes and alleles of valine production

| | Description | | |
|---|---|---|---|
| Gene name | of the coded enzyme or protein | Reference | Access Number |
| aecD | beta C-S Lyase | Rossol et al., Journal | M89931 |
| | EC 2.6.1.1 | of Bacteriology | |
| | (beta C-S lyase) | 174(9): 2968-77 (1992) | |
| ccpA1 | Catabolite Control | WO0100844 | AX065267 |
| | Protein | EP1108790 | AX127147 |
| | (catabolite control | | |
| | protein A1) | | |
| ccpA2 | Catabolite Control | WO0100844 | AX065267 |
| | Protein | EP1108790 | AX121594 |
| | (catabolite control | | |
| | protein A2) | | |
| citA | Sensor Kinase CitA | EP1108790 | AX120161 |
| | (sensor kinase CitA) | | |
| citB | Transcription Regulator | EP1108790 | AX120163 |
| | CitB | | |
| | (transcription regulator | | |
| | CitB) | | |
| citE | Citrate Lyase | WO0100844 | AX065421 |
| | EC 4.1.3.6 | EP1108790 | AX127146 |
| | (citrate lyase) | | |
| ddh | Diaminopimelate | Ishino et al., Nucleic | S07384 |
| | Dehydrogenase | Acids Research 15: 3917 | AX127152 |
| | EC 1.4.1.16 | (1987) | |
| | (diaminopimelate | EP1108790 | |
| | dehydrogenase) | | |
| gluA | Glutamate Transport ATP- | Kronemeyer et al., | X81191 |
| | binding Protein | Journal of Bacteriology | |
| | (glutamate transport ATP- | 177(5): 1152-8 (1995) | |
| | binding protein) | | |
| gluB | Glutamate-binding Protein | Kronemeyer et al., | X81191 |
| | (glutamate-binding | Journal of Bacteriology | |
| | protein) | 177(5): 1152-8 (1995) | |
| gluC | Glutamate Transport | Kronemeyer et al., | X81191 |
| | Permease | Journal of Bacteriology | |
| | (glutamate transport | 177(5): 1152-8 (1995) | |
| | system permease) | | |
| gluD | Glutamate Transport | Kronemeyer et al., | X81191 |
| | Permease | Journal of Bacteriology | |
| | (glutamate transport | 177(5): 1152-8 (1995) | |
| | system permease) | | |
| glyA | Glycine | WO0100843 | AX063861 |
| | Hydroxymethyltransferase | | AF327063 |
| | EC 2.1.2.1 | | |
| | (glycine | | |
| | hydroxymethyltransferase) | | |
| ilvA | Threonine Dehydratase | Möckel et al., Journal | A47044 |
| | EC 4.2.1.16 | of Bacteriology 174 | L01508 |
| | (threonine dehydratase) | (24), 8065-8072 (1992) | AX127150 |
| | | EP1108790 | |
| luxR | Transcription Regulator | WO0100842 | AX065953 |
| | LuxR | EP1108790 | AX123320 |
| | (transcription regulator | | |
| | LuxR) | | |
| lysR1 | Transcription Regulator | EP1108790 | AX064673 |
| | LysR1 | | AX127144 |
| | (transcription regulator | | |
| | LysR1) | | |
| lysR2 | Transcription Activator | EP1108790 | AX123312 |
| | LysR2 | | |
| | (transcription regulator | | |
| | LysR2) | | |
| lysR3 | Transcription Regulator | WO0100842 | AX065957 |
| | LysR3 | EP1108790 | AX127150 |
| | (transcription regulator | | |
| | LysR3) | | |
| panB | Ketopantoate | U.S. Pat. No. 6,177,264 | X96580 |
| | Hydroxymethyltransferase | | |
| | EC 2.1.2.11 | | |
| | (ketopantoate | | |
| | hydroxymethyltransferase) | | |
| panC | Pantothenate Synthetase | U.S. Pat. No. 6,177,264 | X96580 |
| | EC 6.3.2.1 | | |
| | (pantothenate synthetase) | | |
| poxB | Pyruvate Oxidase | WO0100844 | AX064959 |
| | EC 1.2.3.3 | EP1096013 | AX137665 |
| | (pyruvate oxidase) | | |
| zwa2 | Cell Growth Factor 2 | EP1106693 | AX113822 |
| | (growth factor 2) | EP1108790 | AX127146 |

The invention accordingly also provides a process for the production of coryneform bacteria which produce L-valine, characterized in that
a) the nucleotide sequence of a desired ORF, gene or allele of valine production, optionally including the expression and/or regulation signals, is isolated
b) at least two copies of the nucleotide sequence of the ORF, gene or allele of valine production are arranged in a row, preferably in tandem arrangement
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired open ORF, gene or allele of valine production at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and optionally
f) at least a third copy of the open reading frame (ORF), gene or allele of valine production in question is introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

The invention also provides coryneform bacteria, in particular of the genus Corynebacterium, which produce L-tryptophane, characterized in that
a) instead of the singular copy of an open reading frame (ORF), a gene or allele of tryptophane production naturally present at the particular desired site (locus), these have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
b) optionally have at least a third copy of the open reading frame (ORF), gene or allele of tryptophane production mentioned at a further gene site, no nucleotide sequence which is capable of/enables episomal replication in microorganisms, no nucleotide sequence which is capable of/enables transposition and no nucleotide sequence which imparts resistance to antibiotics being present at the further gene site.

The invention also furthermore provides a process for the preparation of L-tryptophane, which comprises the following steps:
a) fermentation of coryneform bacteria, in particular of the genus Corynebacterium, which
   iii) instead of the singular copy of an open reading frame (ORF), gene or allele of tryptophane production present at the particular desired site (locus), have at least two copies of the open reading frame (ORF), gene or allele in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and
   iv) optionally have at least a third copy of the open reading frame (ORF), gene or allele of tryptophane production in question at a further gene site, no nucleotide sequence which is capable of/enables episomal replication in microorganisms, no nucleotide sequence which is capable of/enables transposition and no nucleotide sequence which imparts resistance to antibiotics being present at the further gene site,
      under conditions which allow expression of the said open reading frames (ORFs), genes or alleles,
b) concentration of the L-tryptophane in the fermentation broth,
c) isolation of the L-tryptophane from the fermentation broth, optionally
d) with constituents from the fermentation broth and/or the biomass to the extent of > (greater than) 0 to 100%.

A “copy of an open reading frame (ORF), gene or allele of tryptophane production” is to be understood as meaning all the, preferably endogenous, open reading frames, genes or alleles of which enhancement/over-expression can have the effect of improving tryptophane production.

These include, inter alia, the following open reading frames, genes or alleles: aroA, aroB, aroC, aroD, aroE, aroG, aroK, cstA, eno, gap, gap2, gnd, ppsA, rpe, serA, serB, serC, tal, thyA, tkt, tpi, trpA, trpB, trpC, trpD optionally comprising at least one of the amino acid exchanges selected from the group consisting of A215T (exchange of alanine at position 215 against threonine), D138A (exchange of aspartic acid at position 138 against alanine), S149F (exchange of serine at position 149 against phenylalanine) and A162E (exchange of alanine at position 162 against glutamic acid), trpE, trpE^{FBR} comprising e.g. the amino acid exchange S38R (exchange of serine at position 38 against arginine), trpG, trpL optionally comprising the mutation W14*, zwa1, zwf optionally comprising the amino acid exchange A213T (exchange of alanine at position 213 against threonine). These are summarized and explained in Table 10. These include in particular the tryptophane operon comprising trpL, trpE, trpG, trpD, trpC and trpA. Furthermore these include in particular a trpE^{FBR} allele which codes for a tryptophane-resistant anthranilate synthase.

The at least third, optionally fourth or fifth copy of the open reading frame (ORF), gene or allele of tryptophane production in question can be integrated at a further site. The following open reading frames, genes or nucleotide sequences, inter alia, can be used for this: ccpA1, ccpA2, citA, citB, citE, cysE, gluA, gluB, gluC, gluD, glyA, luxR, luxS, lysR1, lysR2, lysR3, menE, pgi, pheA, poxB and zwa2. These are summarized and explained in Table 11. Intergenic regions in the chromosome, that is to say nucleotide sequences without a coding function, can furthermore be used. Finally, prophages or defective phages or DNA coding for phage components contained in the chromosome can be used for this.

TABLE 10
Open reading frames, genes and alleles of tryptophane
production

| | Description of the coded enzyme or | | Access- |
|---|---|---|---|
| Gene name | protein | Reference | Number |
| aroA | Enolpyruvylshikimate Phosphate | O'Donohue et | AF114233 |
| | Synthase | al., NCBI | |
| | EC 2.5.1.19 | | |
| | (enolpyruvylshikimate 3-phosphate | | |
| | synthase) | | |
| aroB | Dehydroquinate Synthetase | Burke et al., | AF124600 |
| | EC 4.6.1.3 | NCBI | |
| | (dehydroquinate synthetase) | | |
| aroC | Chorismate Synthase | Burke et al., | AF124600 |
| | EC 4.6.1.4 | NCBI | |
| | (chorismate synthase) | | |
| aroD | Dehydroquinate Dehydratase | Joy et al., | AF124518 |
| | EC 4.2.1.10 | NCBI | |
| | (dehydroquinate dehydratase) | | |
| aroE | Shikimate Dehydrogenase | Joy et al., | AF124518 |
| | EC 1.1.1.25 | NCBI | |
| | (shikimate dehydrogenase) | | |
| aroG | Dehydro-3-Deoxyphosphoheptonate | Chen et al., | L07603 |
| | Aldolase | FEMS | |
| | EC4.1.2.15 | Microbioliology | |
| | (dehydro-3-deoxyphosphoheptonate | Letters | |
| | aldolase) | 107: 223-230 | |
| | | (1993). | |
| aroK | Shikimate Kinase | Burke et al., | AF124600 |
| | EC 2.7.1.71 | NCBI | |
| | (shikimate kinase) | | |
| cstA | Carbon Starvation Protein A | EP1108790 | AX120811 |
| | (carbon starvation protein A) | WO0100804 | AX066109 |
| eno | Enolase | EP1108790 | AX127146 |
| | EC 4.2.1.11 | WO0100844 | AX064945 |
| | (enolase) | EP1090998 | AX136862 |
| | | Hermann et al., | |
| | | Electrophoresis | |
| | | 19: 3217-3221 | |
| | | (1998) | |
| gap | Glyceraldehyde-3-Phosphate | EP1108790 | AX127148 |
| | Dehydrogenase | WO0100844 | AX064941 |
| | EC 1.2.1.12 | Eikmanns et | X59403 |
| | (glyceraldehyde-3-phosphate | al., Journal of | |
| | dehydrogenase) | Bacteriology | |
| | | 174: 6076-6086 | |
| | | (1992) | |
| gap2 | Glyceraldehyde-3-Phosphate | EP1108790 | AX127146 |
| | Dehydrogenase | WO0100844 | AX064939 |
| | EC 1.2.1.12 | | |
| | (glyceraldehyde-3-phosphate | | |
| | dehydrogenase 2) | | |
| gnd | 6-Phosphogluconate Dehydrogenase | EP1108790 | AX127147 |
| | EC 1.1.1.44 | | AX121689 |
| | (6-phosphogluconate dehydrogenase) | WO0100844 | AX065125 |
| ppsA | Phosphoenolpyruvate Synthetase | EP1108790 | AX127144 |
| | Ec 2.7.9.2 | | AX120700 |
| | (phosphoenolpyruvate-synthase) | | |
| rpe | Ribulose-Phosphate Epimerase | EP1108790 | AX127148 |
| | EC 5.1.3.1 | | AX121852 |
| | (ribulose-phosphate-epimerase) | | |
| serA | Phosphoglycerate Dehydrogenase | EP1108790 | AX127147 |
| | EC1.1.1.95 | | AX121499 |
| | (phosphoglycerate-dehydrogenase) | | |
| serB | Phosphoserine Phosphatase | EP1108790 | AX127144 |
| | EC 3.1.3.3 | | AX120551 |
| | (phosphoserine phosphatase) | | |
| serC | Phosphoserine Aminotransferase | EP1108790 | AX127145 |
| | EC 2.6.1.52 | | AX121012 |
| | (phosphoserine aminotransferase) | | |
| tal | Transaldolase | WO0104325 | AX076272 |
| | EC 2.2.1.2 | | |
| | (transaldolase) | | |
| thyA | Thymidylate Synthase | EP1108790 | AX121026 |
| | EC 2.1.1.45 | | AX127145 |
| | (thymidylate synthase) | | |
| tkt | Transketolase | Ikeda et al., | AB023377 |
| | EC 2.2.1.1 | NCBI | |
| | (transketolase) | | |
| tpi | Triose-phosphate Isomerase | Eikmanns, | X59403 |
| | EC 5.3.1.1 | Journal of | |
| | (triose-phosphate isomerase) | Bacteriology | |
| | | 174: 6076-6086 | |
| | | (1992) | |
| trpA | Tryptophane Synthase (alpha Kette) | Matsui et al., | X04960 |
| | EC 4.2.1.20 | Nucleic Acids | |
| | (tryptophan synthase (alpha chain)) | Research | |
| | | 14: 10113-10114 | |
| | | (1986) | |
| trpB | Tryptophane Synthase (beta Kette) | Matsui et al., | X04960 |
| | EC 4.2.1.20 | Nucleic Acids | |
| | (tryptophan synthase (beta chain)) | Research | |
| | | 14: 10113-10114 | |
| | | (1986) | |
| trpC | Phosphoribosylanthranilate | Matsui et al., | X04960 |
| | Isomerase | Nucleic Acids | |
| | EC 5.3.1.24 | Research | |
| | (phosphoribosylanthranilate | 14: 10113-10114 | |
| | isomerase) | (1986) | |
| trpD | Anthranilate | Matsui et al., | X04960 |
| | Phosphoribosyltransferase | Nucleic Acids | |
| | EC 2.4.2.18 | Research | |
| | (anthranilate | 14: 10113-10114 | |
| | phosphoribosyltransferase) | (1986) | |
| trpD | Anthranilate | O'Gara et al., | |
| A125T, | Phosphoribosyltransferase | Applied and | |
| D138A, | EC 2.4.2.18 | Environmental | |
| S149F, | anthranilate | Microbiology | |
| A162E | (phosphoribosyltransferase) | 61: 4477-4479 | |
| | amino acid exchanges A125T, D138A, | (1995) | |
| | S149F, A162E | | |
| trpE | Anthranilate Synthase Komponente I | Matsui et al., | X04960 |
| | EC 4.1.3.27 | Nucleic Acids | |
| | (anthranilate synthase component I) | Research | |
| | | 14: 10113-10114 | |
| | | (1986) | |
| trpE | Anthranilat Synthase Component I | Matsui et al., | |
| fbr | feedback resistent | Journal of | |
| | EC 4.1.3.27 | Bacteriology | |
| | (anthranilate synthase component I | 169: 5330-5332 | |
| | feedback resistant) | (1987) | |
| | amino acid exchange S38R | | |
| trpG | Anthranilate Synthase Komponente II | Matsui et al., | X04960 |
| | EC 4.1.3.24 | Nucleic Acids | |
| | (anthranilate synthase component | Research | |
| | II) | 14: 10113-10114 | |
| | | (1986) | |
| trpL | Trp Operon Leader Peptide | Matsui et al., | X04960 |
| | (trp operon leader peptide) | Nucleic Acids | |
| | | Research | |
| | | 14: 10113-10114 | |
| | | (1986) | |
| trpL | Trp Operon Leaderpeptid | Herry et al., | |
| W14* | (trp operon leader peptide | Applied and | |
| | mutation W14*) | Environmental | |
| | | Microbiology | |
| | | 59: 791-799 | |
| | | (1993) | |
| zwa1 | Cell Growth Factor 1 | EP1111062 | AX133781 |
| | (growth factor 1) | | |
| zwf | Glucose-6-phosphatl-1-Dehydrogenase | EP1108790 | AX127148 |
| | EC 1.1.1.49 | | AX121827 |
| | (glucose-6-phosphate-1- | WO0104325 | AX076272 |
| | dehydrogenase) | | |
| zwf | Glucose-6-phosphate-1-Dehydrogenase | EP1108790 | |
| A213T | EC 1.1.1.49 | | |
| | (glucose-6-phosphate-1- | | |
| | dehydrogenase) | | |
| | amino acid exchange A213T | | |

TABLE 11
Further gene sites for integration of open reading frames,
genes and alleles of tryptophane production

| | Description | | |
|---|---|---|---|
| Gene name | of the coded enzyme or protein | Reference | Access Number |
| ccpA1 | Catabolite Control | WO0100844 | AX065267 |
| | Protein | EP1108790 | AX127147 |
| | (catabolite control | | |
| | protein A1) | | |
| ccpA2 | Catabolite Control | WO0100844 | AX065267 |
| | Protein | EP1108790 | AX121594 |
| | (catabolite control | | |
| | protein A2) | | |
| citA | Sensor-Kinase CitA | EP1108790 | AX120161 |
| | (sensor kinase CitA) | | |
| citB | Transcription Regulator | EP1108790 | AX120163 |
| | CitB | | |
| | (transcription regulator | | |
| | CitB) | | |
| citE | Citrate-Lyase | WO0100844 | AX065421 |
| | EC 4.1.3.6 | EP1108790 | AX127146 |
| | (citrate lyase) | | |
| cysE | Serine O- | EP1108790 | AX122902 |
| | Acetyltransferase | | |
| | EC 2.3.1.30 | | |
| | (serine O- | | |
| | acetyltransferase) | | |
| gluA | Glutamate Transport ATP- | Kronemeyer et al., | X81191 |
| | binding Protein | Journal of Bacteriology | |
| | (glutamate transport ATP- | 177(5): 1152-8 (1995) | |
| | binding protein) | | |
| gluB | Glutamate-binding Protein | Kronemeyer et al., | X81191 |
| | (glutamate binding | Journal of Bacteriology | |
| | protein) | 177(5): 1152-8 (1995) | |
| gluC | Glutamate Transport | Kronemeyer et al., | X81191 |
| | Permease | Journal of Bacteriology | |
| | (glutamate transport | 177(5): 1152-8 (1995) | |
| | system permease) | | |
| gluD | Glutamate Transport | Kronemeyer et al., | X81191 |
| | Permease | Journal of Bacteriology | |
| | (glutamate transport | 177(5): 1152-8 (1995) | |
| | system permease) | | |
| glyA | glycine | JP1997028391 | E12594 |
| | hydroxymethyltransferase | | |
| | EC 2.1.2.1 | | |
| | (glycine | | |
| | hydroxymethyltransferase) | | |
| luxR | Transkription Regulator | WO0100842 | AX065953 |
| | LuxR | EP1108790 | AX123320 |
| | (transcription regulator | | |
| | LuxR) | | |
| luxS | Histidine Kinase LuxS | EP1108790 | AX123323 |
| | (histidine kinase LuxS) | | AX127153 |
| lysR1 | Transkription Regulator | EP1108790 | AX064673 |
| | LysR1 | | AX127144 |
| | (transcription regulator | | |
| | LysR1) | | |
| lysR2 | Transkription Activator | EP1108790 | AX123312 |
| | LysR2 | | |
| | (transcription regulator | | |
| | LysR2) | | |
| lysR3 | Transkription Regulator | WO0100842 | AX065957 |
| | LysR3 | EP1108790 | AX127150 |
| | (transcription regulator | | |
| | LysR3) | | |
| menE | O-Succinylbenzoic acid- | WO0100843 | AX064599 |
| | CoA-Ligase | EP1108790 | AX064193 |
| | EC 6.2.1.26 | | AX127144 |
| | (O-succinylbenzoate-CoA | | |
| | ligase) | | |
| pgi | Glucose-6-Phosphate- | EP1087015 | AX136015 |
| | Isomerase | EP1108790 | AX127146 |
| | EC 5.3.1.9 | | |
| | (glucose-6-phosphate | | |
| | isomerase) | | |
| pheA | Prephenate Dehydratase | Follettie et al., | M13774 |
| | EC 4.2.1.51 | Journal of Bacteriology | |
| | (prephenate dehydratase) | 167: 695-702(1986) | |
| poxB | Pyruvate-Oxidase | WO0100844 | AX064959 |
| | EC 1.2.3.3 | EP1096013 | AX137665 |
| | (pyruvate oxidase) | | |
| zwa2 | Cell Growth Factor 2 | EP1106693 | AX113822 |
| | (growth factor 2) | EP1108790 | AX127146 |

The invention accordingly also provides a process for the production of coryneform bacteria which produce L-tryptophane, characterized in that
a) the nucleotide sequence of a desired ORF, gene or allele of tryptophane production, optionally including the expression and/or regulation signals, is isolated
b) at least two copies of the nucleotide sequence of the ORF, gene or allele of tryptophane production are arranged in a row, preferably in tandem arrangement
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired open ORF, gene or allele of tryptophane production at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and optionally
   at least a third copy of the open reading frame (ORF), gene or allele of tryptophane production in question is introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

TABLE 12
Intergenic regions as target sites for integration of open
reading frames, genes and alleles

| | | Position of | Position of |
|---|---|---|---|
| | Access | sequence | sequence |
| Reference | number | start | end |
| EP1108790 | AX120085 | 192176 | 194501 |
| EP1108790 | AX127145 | 235840 | 237311 |
| EP1108790 | AX127145 | 236096 | 237311 |
| EP1108790 | AX127148 | 322628 | 330877 |
| EP1108790 | AX127148 | 334045 | 336467 |
| EP1108790 | AX127148 | 289565 | 291841 |
| EP1108790 | AX127149 | 154823 | 161111 |
| EP1108790 | AX127149 | 190088 | 193497 |
| EP1108790 | AX127149 | 27398 | 28707 |
| EP1108790 | AX127149 | 61478 | 62944 |
| EP1108790 | AX127149 | 116234 | 117561 |
| EP1108790 | AX127149 | 140847 | 144605 |
| EP1108790 | AX127150 | 113274 | 114324 |
| EP1108790 | AX127152 | 244281 | 246403 |

TABLE 13
Target sites coding for phages or phage components suitable
for integration of open reading frames, genes and alleles

| | | Position of | Position of |
|---|---|---|---|
| | Access | sequence | Sequence |
| Reference | number | start | end |
| EP1108790 | AX127149 | 50474 | 51049 |
| EP1108790 | AX127149 | 67886 | 68587 |
| EP1108790 | AX127151 | 72893 | 73480 |
| EP1108790 | AX127149 | 88231 | 89445 |
| EP1108790 | AX127148 | 139781 | 140155 |
| EP1108790 | AX127148 | 140546 | 141001 |
| EP1108790 | AX127149 | 194608 | 195294 |
| EP1108790 | AX127147 | 200185 | 200940 |
| EP1108790 | AX127147 | 208157 | 208450 |
| EP1108790 | AX127149 | 269616 | 269948 |
| EP1108790 | AX127148 | 336468 | 338324 |
| EP1108790 | AX127148 | 342235 | 342681 |
| EP1108790 | AX127148 | 343518 | 345356 |
| EP1108790 | AX127148 | 345872 | 346207 |

During work on the present invention, it was possible to incorporate two copies, arranged in tandem, of an lysC^{FBR} allele at the lysC gene site of Corynebacterium glutamicum such that no nucleotide sequence which is capable of/enables episomal replication in microorganisms, no nucleotide sequence which is capable of/enables transposition and no nucleotide sequence which imparts resistance to antibiotics remain at the lysC gene site. Such a strain is, for example, the strain DSM13992lysC^{FBR}::lysC^{FBR}.

The plasmid pK18mobsacB2xlysCSma2/1, with the aid of which two copies of an lysC^{FBR} allele can be incorporated into the lysC gene site of Corynebacterium glutamicum, is shown in FIG. 1.

During work on the present invention, it was furthermore possible to incorporate two copies, arranged in tandem, of the lysE gene at the lysE gene site of Corynebacterium glutamicum such that no nucleotide sequence which is capable of/enables episomal replication in microorganisms, no nucleotide sequence which is capable of/enables transposition and no nucleotide sequence which imparts resistance to antibiotics remained at the lysE gene site. Such a strain is, for example, the strain ATCC21513_{—}17lysE::lysE.

A plasmid with the aid of which two copies of an lysE gene can be incorporated into the lysE gene site of Corynebacterium glutamicum is shown in FIG. 2. It carries the name pK18mobsacB2xlysESma1/1.

During work on the present invention, finally, it was possible to incorporate two copies, arranged in tandem, of the zwa1 gene at the zwa1 gene site of Corynebacterium glutamicum such that no nucleotide sequence which is capable of/enables episomal replication in microorganisms, no nucleotide sequence which is capable of/enables transposition and no nucleotide sequence which imparts resistance to antibiotics remained at the zwa1 gene site. Such a strain is, for example, the strain ATCC21513_{—}17zwa1::zwa1.

A plasmid with the aid of which two copies of a zwa1 gene can be incorporated into the zwa1 gene site of Corynebacterium glutamicum is shown in FIG. 3. It carries the name pK18mobsacBzwa1zwa1.

The coryneform bacteria produced according to the invention can be cultured continuously or discontinuously in the batch process (batch culture) or in the fed batch (feed process) or repeated fed batch process (repetitive feed process) for the purpose of production of chemical compounds. A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid or lactic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired chemical compound has formed. This target is usually reached within 10 hours to 160 hours.

It has been found that the coryneform bacteria according to the invention, in particular the coryneform bacteria which produce L-lysine, have an unexpectedly high stability. They were stable for at least 10-20, 20-30, 30-40, 40-50, preferably at least 50-60, 60-70, 70-80 and 80-90 generations or cell division cycles.

The following microorganisms have been deposited:

The Corynebacterium glutamicum strain DSM13992lysC^{FBR}::lysC^{FBR} was deposited in the form of a pure culture on 5 Jun. 2002 under number DSM15036 at the Deutsche Sammlung für Mikroorganismen und Zellkulturen (DSMZ, Braunschweig, Germany) in accordance with the Budapest Treaty.

The plasmid pK18mobsacB2xlysCSma2/1 was deposited in the form of a pure culture of the strain E. coli DH5αmcr/pK18mobsacB2xlysCSma2/1 (=DH5alphamcr/pK18mobsacB2xlysCSma2/1) on 20 Apr. 2001 under number DSM14244 at the Deutsche Sammlung für Mikroorganismen und Zellkulturen (DSMZ, Braunschweig, Germany) in accordance with the Budapest Treaty.

The Corynebacterium glutamicum strain ATCC21513_{—}17lysE::lysE was deposited in the form of a pure culture on 5 Jun. 2002 under number DSM15037 at the Deutsche Sammlung für Mikroorganismen und Zellkulturen (DSMZ, Braunschweig, Germany) in accordance with the Budapest Treaty.

The Corynebacterium glutamicum strain ATCC21513_{—}17zwa1::zwa1 was deposited in the form of a pure culture on 5 Jun. 2002 under number DSM15038 at the Deutsche Sammlung für Mikroorganismen und Zellkulturen (DSMZ, Braunschweig, Germany) in accordance with the Budapest Treaty.

### Example 1

### Generation of a Tandem Duplication of the lysC^{FBR} Allele lysC T311I in the Chromosome of Corynebacterium glutamicum

1.1. Construction of the Tandem Vector pK18mobsacB2xlysCSma2/1

From the Corynebacterium glutamicum strain DSM13994, chromosomal DNA is isolated by the conventional methods (Eikmanns et al., Microbiology 140: 1817-1828 (1994)).

The strain DSM13994 was produced by multiple, non-directed mutagenesis, selection and mutant selection from C. glutamicum ATCC13032. The strain is resistant to the lysine analogue S-(2-aminoethyl)-L-cysteine and has a feed back-resistant aspartate kinase which is insensitive to inhibition by a mixture of lysine and threonine (in each case 25 mM). The nucleotide sequence of the lysC^{FBR} allele is shown as SEQ ID NO:3. It is also called lysC T311I in the following. The amino acid sequence of the aspartate kinase protein coded is shown as SEQ ID NO:4. A pure culture of this strain was deposited on 16 Jan. 2001 at the Deutsche Sammlung für Mikroorganismen and Zellkulturen (DSMZ, Braunschweig, Germany) in accordance with the Budapest Treaty.

With the aid of the polymerase chain reaction, a DNA section which carries the lysC gene or allele is amplified. On the basis of the sequence of the lysC gene known for C. glutamicum (Kalinowski et al., Molecular Microbiology, 5 (5), 1197-1204 (1991); Accession Number X57226), the following primer oligonucleotides were chosen for the PCR:

lysC1beg (SEQ ID No: 15):
5′ TA(G GAT CC)T CCG GTG TCT GAC CAC GGT G 3′
lysC2end: (SEQ ID NO: 16):
5′ AC(G GAT CC)G CTG GGA AAT TGC GCT CTT CC 3′

The primers shown are synthesized by MWG Biotech and the PCR reaction is carried out by the standard PCR method of Innis et al. (PCR Protocols. A Guide to Methods and Applications, 1990, Academic Press). The primers allow amplification of a DNA section of approx. 1.7 kb in length, which carries the lysC gene or allele. The primers moreover contain the sequence for a cleavage site of the restriction endonuclease BamHI, which is marked by parentheses in the nucleotide sequence shown above.

The amplified DNA fragment of approx. 1.7 kb in length which carries the lysC^{FBR} allele lysC T311I of the strain DSM13994 is identified by electrophoresis in a 0.8% agarose gel, isolated from the gel and purified by conventional methods (QIAquick Gel Extraction Kit, Qiagen, Hilden).

Ligation of the fragment is then carried out by means of the Topo TA Cloning Kit (Invitrogen, Leek, The Netherlands, Cat. Number K4600-01) in the vector pCRII-TOPO. The ligation batch is transformed in the E. coli strain TOP10 (Invitrogen, Leek, The Netherlands). Selection of plasmid-carrying cells is made by plating out the transformation batch on kanamycin (50 mg/l)-containing LB agar with X-Gal (5-bromo-4-chloro-3-indolyl β-D-galactopyranoside, 64 mg/l).

The plasmid obtained is checked by means of restriction cleavage, after isolation of the DNA, and identified in agarose gel. The resulting plasmid is called pCRIITOPolysC.

The nucleotide sequence of the amplified DNA fragment or PCR product is determined by the dideoxy chain termination method of Sanger et al. (Proceedings of the National Academy of Sciences USA, 74:5463-5467 (1977)) using the “ABI Prism 377” sequencing apparatus of PE Applied Biosystems (Weiterstadt, Germany). The sequence of the coding region of the PCR product is shown in SEQ ID No:3.

The amino acid sequence of the associated aspartate kinase protein is shown in SEQ ID NO:4.

The base thymine is found at position 932 of the nucleotide sequence of the coding region of the lysC^{FBR} allele of strain DSM13994 (SEQ ID NO:3). The base cytosine is found at the corresponding position of the wild-type gene (SEQ ID NO:1).

The amino acid isoleucine is found at position 311 of the amino acid sequence of the aspartate kinase protein of strain DSM13994 (SEQ ID No:4). The amino acid threonine is found at the corresponding position of the wild-type protein (SEQ ID No:2).

The lysC allele, which contains the base thymine at position 932 of the coding region and accordingly codes for an aspartate kinase protein which contains the amino acid isoleucine at position 311 of the amino acid sequence, is called the lysC^{FBR} allele lysC T311I in the following.

The plasmid pCRIITOPolysC, which carries the lysC^{FBR} allele lysC T311I, was deposited in the form of a pure culture of the strain E. coli TOP 10/pCRIITOPolysC under number DSM14242 on 20 Apr. 2001 at the Deutsche Sammlung für Mikroorganismen and Zellkulturen (DSMZ=German Collection of Microorganisms and Cell Cultures, Braunschweig, Germany) in accordance with the Budapest Treaty.

Plasmid DNA was isolated from the strain DSM14242, which carries the plasmid pCRIITOPolysC, and cleaved with the restriction enzyme BamHI (Amersham-Pharmacia, Freiburg, Germany), after separation in an agarose gel (0.8%) the lysC^{FBR}-containing DNA fragment approx. 1.7 kb long is isolated from the agarose gel with the aid of the QIAquick Gel Extraction Kit (Qiagen, Hilden, Germany), and the overhanging ends are completed with Klenow polymerase (Boehringer Mannheim) and employed for ligation with the mobilizable cloning vector pK18mobsacB described by Schäfer et al., Gene, 14, 69-73 (1994). This is cleaved beforehand with the restriction enzyme SmaI and dephosphorylated with alkaline phosphatase (Alkaline Phosphatase, Boehringer Mannheim), mixed with the lysC^{FBR}-containing fragment of approx. 1.7 kb and the mixture is treated with T4 DNA Ligase (Amersham-Pharmacia, Freiburg, Germany).

The E. coli strain DH5α (Grant et al.; Proceedings of the National Academy of Sciences USA, 87 (1990) 4645-4649) is then transformed with the ligation batch (Hanahan, In. DNA Cloning. A Practical Approach. Vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzyme HindIII and subsequent agarose gel electrophoresis. The plasmid is called pK18mobsacB1xlysCSma2.

In a second step, the plasmid pCRII-TOPOlysC is in turn cleaved with the restriction enzyme BamHI (Amersham-Pharmacia, Freiburg, Germany), after separation in an agarose gel (0.8%) the lysC^{FBR}-containing fragment of approx. 1.7 kb was isolated from the agarose gel with the aid of the QIAquick Gel Extraction Kit (Qiagen, Hilden, Germany) and employed for ligation with the vector pK18mobsacB1xlysCSma2 described in this Example. This is cleaved beforehand with the restriction enzyme BamHI and dephosphorylated with alkaline phosphatase (Alkaline Phosphatase, Boehringer Mannheim), mixed with the lyse-containing fragment of approx. 1.7 kb and the mixture is treated with T4 DNA Ligase (Amersham-Pharmacia, Freiburg, Germany).

The E. coli strain DH5α (Grant et al.; Proceedings of the National Academy of Sciences USA, 87 (1990) 4645-4649) is then transformed with the ligation batch (Hanahan, In. DNA Cloning. A Practical Approach. Vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzyme HindIII and subsequent agarose gel electrophoresis. The plasmid is called pK18mobsacB2xlysCSma2/1. A map of the plasmid is shown in FIG. 1.

The plasmid pK18mobsacB2xlysCSma2/1 was deposited in the form of a pure culture of the strain E. coli DH5αmcr/pK18mobsacB2xlysCSma2/1 (=DH5alphamcr/pK18mobsacB2xlysCSma2/1) on 20 Apr. 2001 under number DSM14244 at the Deutsche Sammlung für Mikroorganismen and Zellkulturen (DSMZ, Braunschweig, Germany) in accordance with the Budapest Treaty.

1.2. Generation of a Tandem Duplication of the lysC^{FBR} Allele lysC T311I in C. glutamicum Strain DSM13992

The vector pK18mobsacB2xlysCSma2/1 mentioned in Example 1.1 is transferred by a modified protocol of Schäfer et al. (1990 Journal of Microbiology 172: 1663-1666) into the C. glutamicum strain DSM13992.

The Corynebacterium glutamicum strain DSM13992 was produced by multiple, non-directed mutagenesis, selection and mutant selection from C. glutamicum ATCC13032. The strain is resistant to the antibiotic streptomycin and phenotypically resistant to the lysine analogue S-(2-aminoethyl)-L-cysteine. However, the strain has a wild-type aspartate kinase (see SEQ ID NO:1 and 2), which is sensitive to inhibition by a mixture of lysine and threonine (in each case 25 mM). A pure culture of this strain was deposited on 16 Jan. 2001 at the Deutsche Sammlung für Mikroorganismen and Zellkulturen (DSMZ, Braunschweig, Germany) in accordance with the Budapest Treaty.

The vector pK18mobsacB2xlysCSma2/1 cannot replicate independently in DSM13992 and is retained in the cell only if it has integrated into the chromosome.

Selection of clones with integrated pK18mobsacB2xlysCSma2/1 is carried out by plating out the conjugation batch on LB agar (Sambrook et al., Molecular Cloning: A Laboratory Manual. 2^{nd} Ed., Cold Spring Harbor, N.Y., 1989), which was supplemented with 15 mg/l kanamycin and 50 mg/l nalidixic acid. Clones which have grown on are plated out on LB agar plates with 25 mg/l kanamycin and incubated for 16 hours at 33° C. To achieve excision of the plasmid with only one copy of the lysC gene, the clones are cultured on LB agar with 10% sucrose, after incubation for 16 hours in LB liquid medium. The plasmid pK18mobsacB contains a copy of the sacB gene, which converts sucrose into levan sucrase, which is toxic to C. glutamicum.

Only those clones in which the pK18mobsacB2xlysCSma2/1 integrated has been excised again therefore grow on LB agar with sucrose. Approximately 40 to 50 colonies are tested for the phenotype “growth in the presence of sucrose” and “non-growth in the presence of kanamycin”. During the excision, either two copies of the lysC gene or only one can be excised together with the plasmid.

To demonstrate that two copies of lysC have remained in the chromosome, approximately 20 colonies which show the phenotype “growth in the presence of sucrose” and “non-growth in the presence of kanamycin” are investigated with the aid of the polymerase chain reaction by the standard PCR method of Innis et al. (PCR Protocols. A Guide to Methods and Applications, 1990, Academic Press). A DNA fragment which carries the lysC gene and surrounding regions is amplified here from the chromosomal DNA of the colonies. The following primer oligonucleotides are chosen for the PCR.

lysCK1 (SEQ ID NO: 5):
5′ TCG GTG TCA TCA GAG CAT TG 3′
lysCK2 (SEQ ID NO: 6):
5′ TCG GTT GCC TGA GTA ATG TC 3′

The primers allow amplification of a DNA fragment approx. 1.9 kb in size in control clones with the original lysC locus. In clones with a second copy of the lysC gene in the chromosome at the lysC locus, DNA fragments with a size of approx. 3.6 kb are amplified.

The amplified DNA fragments are identified by means of electrophoresis in a 0.8% agarose gel. On the basis of the amplified fragment length, a distinction was made between clones with one chromosomal lysC gene copy and clones with two chromosomal lysC gene copies.

10 clones with two complete copies of the lysC gene on the chromosome are investigated with the aid of the LightCycler of Roche Diagnostics (Mannheim, Germany) in order to demonstrate whether the two copies are lysC^{FBR} alleles with the mutation lysC T311I or whether the original wild-type lysC is present alongside an lysC^{FBR} allele lysC T311I. The LightCycler is a combined apparatus of Thermocycler and fluorimeter.

A DNA section approx. 500 by in length which contains the mutation site is amplified in the first phase by means of a PCR (Innis et al., PCR Protocols. A Guide to Methods and Applications, 1990, Academic Press) using the following primer oligonucleotides.

LC-lysC1-fbr (SEQ ID No: 7):
5′ aaccgttctgggtatttccg 3′
LC-lysC2-fbr (SEQ ID No: 8):
5′ tccatgaactctgcggtaac 3′

In the second phase, with two additional oligonucleotides of different lengths and marked with different fluorescent dyestuffs (Lightcycler(LC)-Red640 and fluorescein), which hybridize in the region of the mutation site, the presence of the mutation is detected with the aid of the “Fluorescence Resonance Energy Transfer” method (FRET) using a melting curve analysis (Lay et al., Clinical Chemistry, 43:2262-2267 (1997)).

lysC311-C (SEQ ID No: 9):
5′ LC-Red640-gcaggtgaagatgatgtcggt-(P) 3′
lysC311-A (SEQ ID No: 10):
5′ tcaagatctccatcgcgcggcggccgtcggaacga-
fluorescein 3′

The primers shown are synthesized for the PCR by MWG Biotech and oligonucleotides shown for the hybridization are synthesized by TIB MOLBIOL (Berlin, Germany).

A clone which contains the base thymine at position 932 of the coding regions of the two lysC copies and thus has two lysC^{FBR} alleles lysC T311I was identified in this manner.

The strain was called C. glutamicum DSM13992lysC^{FBR}:lysC^{FBR}.

The strain was deposited as C. glutamicum DSM13992lysC^{FBR}::lysC^{FBR} on 5 Jun. 2002 under number DSM15036 at the Deutsche Sammlung für Mikroorganismen and Zellkulturen (DSMZ, Braunschweig, Germany) in accordance with the Budapest Treaty.

### Example 2

### Generation of a Tandem Duplication of the lysE Gene in the Chromosome of Corynebacterium glutamicum

2.1. Construction of the Tandem Vector pK18mobsacB2xlysESma1/1

Plasmid DNA was isolated from the Escherichia coli strain DSM12871 (EP-A-1067193), which carries the plasmid pEC7lysE.

The plasmid contains the lysE gene which codes for lysine export. A pure culture of this strain was deposited on 10th June 1999 at the Deutsche Sammlung für Mikroorganismen and Zellkulturen (DSMZ, Braunschweig, Germany) in accordance with the Budapest Treaty.

The plasmid pEC71lysE is cleaved with the restriction enzyme BamHI (Amersham-Pharmacia, Freiburg, Germany), after separation in an agarose gel (0.8%) the lysE fragment of approx. 1.1 kb is isolated from the agarose gel with the aid of the QIAquick Gel Extraction Kit (Qiagen, Hilden, Germany), and the overhanging ends are completed with Klenow polymerase (Boehringer Mannheim) and employed for ligation with the mobilizable cloning vector pK18mobsacB described by Schäfer et al., Gene, 14, 69-73 (1994). This is cleaved beforehand with the restriction enzyme SmaI and dephosphorylated with alkaline phosphatase (Alkaline Phosphatase, Boehringer Mannheim), mixed with the lysE fragment of approx. 1.1 kb and the mixture is treated with T4 DNA Ligase (Amersham-Pharmacia, Freiburg, Germany).

The E. coli strain DH5α (Grant et al.; Proceedings of the National Academy of Sciences USA, 87 (1990) 4645-4649) is then transformed with the ligation batch (Hanahan, In. DNA Cloning. A Practical Approach. Vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzymes BamHI and EcoRI and subsequent agarose gel electrophoresis. The plasmid is called pK18mobsacB1xlysESma1.

In a second step, the plasmid pEC7lysE is in turn cleaved with the restriction enzyme BamHI (Amersham-Pharmacia, Freiburg, Germany), after separation in an agarose gel (0.8%) the lysE fragment of approx. 1.1 kb was isolated from the agarose gel with the aid of the QIAquick Gel Extraction Kit (Qiagen, Hilden, Germany) and employed for ligation with the vector pK18mobsacB1xlysESma1 described in this Example. This is cleaved beforehand with the restriction enzyme BamHI and dephosphorylated with alkaline phosphatase (Alkaline Phosphatase, Boehringer Mannheim), mixed with the lysE fragment of approx. 1.1 kb and the mixture is treated with T4 DNA Ligase (Amersham-Pharmacia, Freiburg, Germany).

The E. coli strain DH5α (Grant et al.; Proceedings of the National Academy of Sciences USA, 87 (1990) 4645-4649) is then transformed with the ligation batch (Hanahan, In. DNA Cloning. A Practical Approach. Vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzymes EcoRI and SalI or ScaI and subsequent agarose gel electrophoresis. The plasmid is called pK18mobsacB2xlysESma1/1. A map of the plasmid is shown in FIG. 2.

2.2. Generation of a Tandem Duplication of the lysE Gene in C. glutamicum Strain ATCC21513_{—}17

The vector pK18mobsacB2xlysESma1/1 mentioned in Example 2.1 is transferred by a modified protocol of Schäfer et al. (1990 Journal of Microbiology 172: 1663-1666) into the C. glutamicum strain ATCC21513_{—}17.

The Corynebacterium glutamicum strain ATCC21513_{—}17 was produced by multiple, non-directed mutagenesis, selection and mutant selection from C. glutamicum ATCC21513. The strain is resistant to the lysine analogue S-(2-aminoethyl)-L-cysteine and both leucine- and homoserine-prototrophic.

The vector cannot replicate independently in ATCC21513_{—}17 and is retained in the cell only if it has integrated into the chromosome.

Selection of clones with integrated pK18mobsacB2xlysESma1/1 is carried out by plating out the conjugation batch on LB agar (Sambrook et al., Molecular Cloning: A Laboratory Manual. 2^{nd} Ed., Cold Spring Harbor, N.Y., 1989), which was supplemented with 15 mg/l kanamycin and 50 mg/l nalidixic acid. Clones which have grown on are plated out on LB agar plates with 25 mg/l kanamycin and incubated for 16 hours at 33° C. To achieve excision of the plasmid with only one copy of the lysE gene, the clones are cultured on LB agar with 10% sucrose, after incubation for 16 hours in LB liquid medium. The plasmid pK18mobsacB contains a copy of the sacB gene, which converts sucrose into levan sucrase, which is toxic to C. glutamicum.

Only those clones in which the pK18mobsacB2xlysESma1/1 integrated has been excised again therefore grow on LB agar with sucrose. Approximately 40 to 50 colonies are tested for the phenotype “growth in the presence of sucrose” and “non-growth in the presence of kanamycin”. During the excision, either two copies of the lysE gene or only one can be excised together with the plasmid.

To demonstrate that two copies of lysE have remained in the chromosome, approximately 20 colonies which show the phenotype “growth in the presence of sucrose” and “non-growth in the presence of kanamycin” are investigated with the aid of the polymerase chain reaction by the standard PCR method of Innis et al. (PCR Protocols. A Guide to Methods and Applications, 1990, Academic Press). A DNA fragment which carries the lysE gene and surrounding regions is amplified here from the chromosomal DNA of the colonies. The following primer oligonucleotides are chosen for the PCR.

lysEK-1 (SEQ ID NO: 11):
5′ TGC TTG CAC AAG GAC TTC AC 3′
lysEK-2 (SEQ ID NO: 12):
5′ TAT GGT CCG CAA GCT CAA TG 3′

The primers allow amplification of a DNA fragment approx. 1.2 kb in size in control clones with the original lysE locus. In clones with a second copy of the lysC gene in the chromosome at the lysE locus, DNA fragments with a size of approx. 2.3 kb are amplified.

The amplified DNA fragments are identified by means of electrophoresis in a 0.8% agarose gel. On the basis of the amplified fragment length, a distinction was made between clones with one chromosomal lysE gene copy and clones with two chromosomal lysE gene copies. It could thus be demonstrated that the strain ATCC21513_{—}17 carries two complete copies of the lysE gene on the chromosome.

The strain was called C. glutamicum ATCC21513_{—}17lysE::lysE.

The strain was deposited as C. glutamicum ATCC21513_{—}17lysE::lysE on 5 Jun. 2002 under number DSM15037 at the Deutsche Sammlung für Mikroorganismen und Zellkulturen (DSMZ, Braunschweig, Germany) in accordance with the Budapest Treaty.

### Example 3

### Generation of a Tandem Duplication of the zwa1 Gene in the Chromosome of Corynebacterium glutamicum

3.1. Construction of the Tandem Vector pK18mobsacBzwa1zwa1

Plasmid DNA was isolated from the Escherichia coli strain DSM13115 (EP-A-1111062), which carries the plasmid pCR2.1zwa1exp.

The plasmid contains the zwa1 gene which codes for cell growth factor 1. A pure culture of this strain was deposited on 19 Oct. 1999 at the Deutsche Sammlung far Mikroorganismen und Zellkulturen (DSMZ, Braunschweig, Germany) in accordance with the Budapest Treaty.

The plasmid pCR2.1zwa1exp is cleaved with the restriction enzyme EcoRI (Amersham-Pharmacia, Freiburg, Germany), and after separation in an agarose gel (0.8%) the zwa1 fragment of 1 kb is isolated from the agarose gel with the aid of the QIAquick Gel Extraction Kit (Qiagen, Hilden, Germany) and employed for ligation with the mobilizable cloning vector pK18mobsacB described by Schäfer et al., Gene, 14, 69-73 (1994). This is cleaved beforehand with the restriction enzyme EcoRI and dephosphorylated with alkaline phosphatase (Alkaline Phosphatase, Boehringer Mannheim), mixed with the zwa1 fragment of 1 kb and the mixture is treated with T4 DNA Ligase (Amersham-Pharmacia, Freiburg, Germany).

The E. coli strain DH5α (Grant et al.; Proceedings of the National Academy of Sciences USA, 87 (1990) 4645-4649) is then transformed with the ligation batch (Hanahan, In. DNA Cloning. A Practical Approach. Vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual. 2^{nd} Ed., Cold Spring Harbor, N.Y., 1989), which was supplemented with 25 mg/l kanamycin.

Plasmid DNA is isolated from a transformant with the aid of the QIAprep Spin Miniprep Kit from Qiagen and checked by restriction cleavage with the enzyme NheI and subsequent agarose gel electrophoresis. Checking of the plasmid showed that two zwa1 fragments were cloned simultaneously and in the desired orientation in the cloning vector pK18mobsac.

The plasmid is called pK18mobsacBzwa1zwa1. A map of the plasmid is shown in FIG. 3.

3.2. Generation of a Tandem Duplication of the zwa1 Gene in C. glutamicum Strain ATCC21513_{—}17

The vector pK18mobsacBzwa1zwa1 mentioned in Example 3.1 is transferred by a modified protocol of Schäfer et al. (1990 Journal of Microbiology 172: 1663-1666) into the C. glutamicum strain ATCC21513_{—}17.

The Corynebacterium glutamicum strain ATCC21513_{—}17 was produced by multiple, non-directed mutagenesis, selection and mutant selection from C. glutamicum ATCC21513. The strain is resistant to the lysine analogue S-(2-aminoethyl)-L-cysteine and both leucine- and homoserine-prototrophic.

The vector cannot replicate independently in ATCC21513_{—}17 and is retained in the cell only if it has integrated into the chromosome.

Selection of clones with integrated pK18mobsacBzwa1zwa1 is carried out by plating out the conjugation batch on LB agar (Sambrook et al., Molecular Cloning: A Laboratory Manual. 2^{nd} Ed., Cold Spring Harbor, New York, 1989), which was supplemented with 15 mg/l kanamycin and 50 mg/l nalidixic acid. Clones which have grown on are plated out on LB agar plates with 25 mg/l kanamycin and incubated for 16 hours at 33° C. To achieve excision of the plasmid with only one copy of the zwa1 gene, the clones are cultured on LB agar with 10% sucrose, after incubation for 16 hours in LB liquid medium. The plasmid pK18mobsacB contains a copy of the sacB gene, which converts sucrose into levan sucrase, which is toxic to C. glutamicum.

Only those clones in which the pK18mobsacBzwa1zwa1 integrated has been excised again therefore grow on LB agar with sucrose. Approximately 40 to 50 colonies are tested for the phenotype “growth in the presence of sucrose” and “non-growth in the presence of kanamycin”. During the excision, either two copies of the zwa1 gene or only one can be excised together with the plasmid.

To demonstrate that two copies of zwa1 have remained in the chromosome, approximately 20 colonies which show the phenotype “growth in the presence of sucrose” and “non-growth in the presence of kanamycin” are investigated with the aid of the polymerase chain reaction by the standard PCR method of Innis et al. (PCR Protocols. A Guide to Methods and Applications, 1990, Academic Press). A DNA fragment which carries the zwa1 gene and surrounding regions is amplified here from the chromosomal DNA of the colonies. The following primer oligonucleotides are chosen for the PCR.

zwa1-A2 (SEQ ID NO: 13):
5′ CAC TTG TCC TCA CCA CTT TC 3′
zwa1-E1 (SEQ ID NO: 14):
5′ TTC TAC TGG GCG TAC TTT CG 3′

The primers allow amplification of a DNA fragment approx. 1.3 kb in size in control clones with the original zwa1 locus. In clones with a second copy of the zwa1 gene in the chromosome at the zwa1 locus, DNA fragments with a size of approx. 2.3 kb are amplified.

The amplified DNA fragments are identified by means of electrophoresis in a 0.8% agarose gel. On the basis of the amplified fragment length, a distinction was made between clones with one chromosomal zwa1 gene copy and clones with two chromosomal zwa1 gene copies. It could thus be demonstrated that the strain ATCC21513_{—}17 carries two complete copies of the zwa1 gene on the chromosome.

The strain was called C. glutamicum ATCC21513_{—}17zwa1::zwa1. The strain was deposited as C. glutamicum ATCC21513_{—}17zwa1::zwa1 on 5 Jun. 2002 under number DSM15038 at the Deutsche Sammlung für Mikroorganismen and Zellkulturen (DSMZ, Braunschweig, Germany) in accordance with the Budapest Treaty.

### Example 4

### Preparation of Lysine

The C. glutamicum strains DSM13992lysC^{FBR}::lysC^{FBR}, ATCC21513_{—}17lysE::lysE and ATCC21513_{—}17zwa1::zwa1 obtained in Examples 1 to 3 are cultured in a nutrient medium suitable for the production of lysine and the lysine content in the culture supernatant was determined.

For this, the strains are first incubated on an agar plate for 24 hours at 33° C. Starting from this agar plate culture, a preculture is seeded (10 ml medium in a 100 ml conical flask). The medium MM is used as the medium for the preculture. The preculture is incubated for 24 hours at 33° C. at 240 rpm on a shaking machine. A main culture is seeded from this preculture such that the initial OD (660 nm) of the main culture is 0.1 OD. The Medium MM is also used for the main culture.

Medium MM

| | |
|---|---|
| CSL | 5 g/l |
| MOPS | 20 g/l |
| Glucose (autoclaved separately) | 50 g/l |

### Salts:

| | | |
|---|---|---|
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| CaCO₃ | 25 | g/l |

The CSL (corn steep liquor), MOPS (morpholinopropanesulfonic acid) and the salt solution are brought to pH 7 with aqueous ammonia and autoclaved. The sterile substrate and vitamin solutions, as well as the CaCO₃ autoclaved in the dry state, are then added.

Culturing is carried out in a 10 ml volume in a 100 ml conical flask with baffles. Culturing is carried out at 33° C. and 80% atmospheric humidity.

After 48 hours, the OD is determined at a measurement wavelength of 660 nm with a Biomek 1000 (Beckmann Instruments GmbH, Munich). The amount of lysine formed is determined with an amino acid analyzer from Eppendorf-BioTronik (Hamburg, Germany) by ion exchange chromatography and post-column derivation with ninhydrin detection.

The result of the experiment is shown in Table 10.

TABLE 10

| | OD | Lysine HCl |
|---|---|---|
| Strain | (660 nm) | g/l |
| DSM13992 | 12.8 | 18.9 |
| DSM13992lysC^{FBR}::lysC^{FBR} | 12.0 | 21.6 |
| ATCC21513_17 | 10.4 | 14.0 |
| ATCC21513_17lysE::lysE | 10.0 | 14.3 |
| ATCC21513_17zwal::zwal | 9.9 | 14.6 |

### BRIEF DESCRIPTION OF THE FIGURES

The base pair numbers stated are approximate values obtained in the context of reproducibility of measurements.

FIG. 1: Map of the plasmid pK18mobsacB2xlysCSma2/1.

The abbreviations and designations used have the following meaning:
KmR: Kanamycin resistance gene
HindIII: Cleavage site of the restriction enzyme HindIII
BamHI: Cleavage site of the restriction enzyme BamHI
lysC: lysC^{FBR} allele lysC T311I
sacB: sacB gene
RP4mob: mob region with the replication origin for the transfer (oriT)
oriV: Replication origin V

FIG. 2: Map of the plasmid pK18mobsacB2xlysESma1/1.

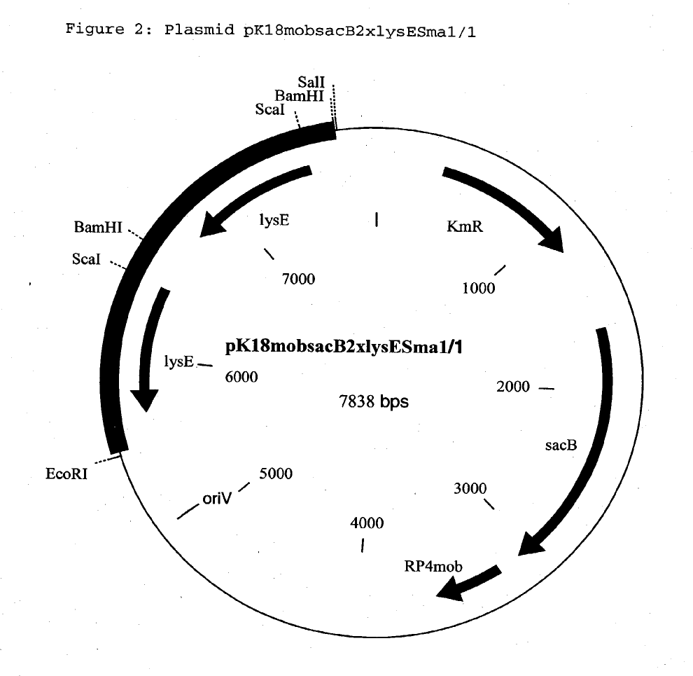

The abbreviations and designations used have the following meaning:
KanR: Kanamycin resistance gene
SalI: Cleavage site of the restriction enzyme SalI
BamHI: Cleavage site of the restriction enzyme BamHI
EcoRI: Cleavage site of the restriction enzyme EcoRI
ScaI: Cleavage site of the restriction enzyme ScaI
lysE: lysE gene
sacB: sacB gene
RP4mob: mob region with the replication origin for the transfer (oriT)
oriV: Replication origin V

FIG. 3: Map of the plasmid pK18mobsacBzwa1zwa1.

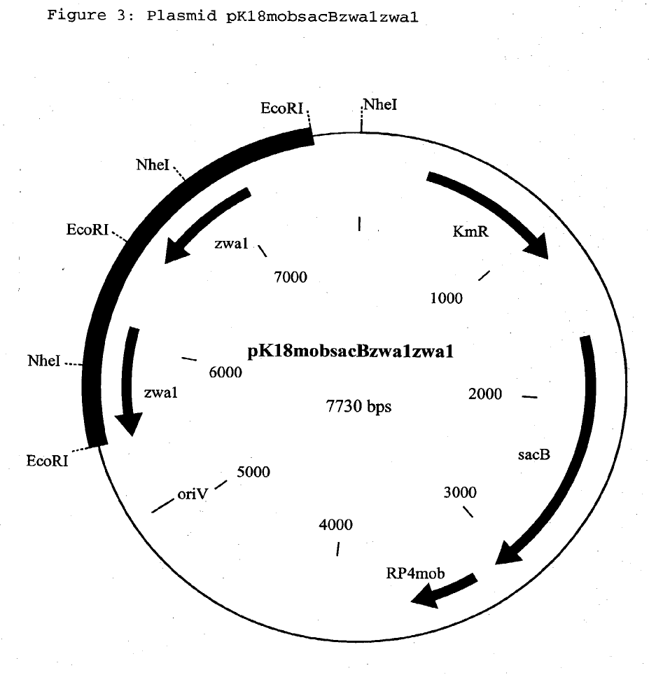

The abbreviations and designations used have the following meaning:
KanR: Kanamycin resistance gene
EcoRI: Cleavage site of the restriction enzyme EcoRI
NheI: Cleavage site of the restriction enzyme NheI
zwa1: zwa1 gene
sacB: sacB gene
RP4mob: mob region with the replication origin for the transfer (oriT)
oriV: Replication origin V

What is claimed is:

1. Coryneform bacteria which produce chemical compounds, wherein instead of the singular copy of an open reading frame (ORF), gene or allele naturally present at the particular desired site (locus), these have at least two copies of the open reading frame (ORF), gene or allele in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these optionally
have at least a third copy of the open reading frame (ORF), gene or allele in question at a further gene site, no nucleotide sequence which is capable of/enables episomal replication in microorganisms, no nucleotide sequence which is capable of/enables transposition and no nucleotide sequence which imparts resistance to antibiotics being present at the further gene site.

2. Coryneform bacteria according to claim 1 which produce chemical compounds, wherein the coryneform bacteria belong to the genus Corynebacterium.

3. Coryneform bacteria of the genus Corynebacterium according to claim 2 which produce chemical compounds, wherein these belong to the species Corynebacterium glutamicum.

4. Coryneform bacteria according to claim 1 which produce chemical compounds, wherein the chemical compound is a compound chosen from the group consisting of L-amino acids, vitamins, nucleosides and nucleotides.

5. Coryneform bacteria according to claim 1 which produce chemical compounds, wherein the chemical compound is one or more L-amino acids chosen from the group consisting of L-aspartic acid, L-asparagine, L-threonine, L-serine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine.

6. Coryneform bacteria according to claim 1 which produce chemical compounds, wherein the chemical compound is the amino acid L-lysine.

7. Coryneform bacteria which produce L-lysine, wherein instead of the singular copy of an open reading frame (ORF), gene or allele of lysine production naturally present at the particular desired site (locus), these have at least two copies of the open reading frame (ORF), gene or allele of lysine production in question, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and in that these
optionally have at least a third copy of the open reading frame (ORF), gene or allele of lysine production in question at a further gene site, no nucleotide sequence which is capable of/enables episomal replication in microorganisms, no nucleotide sequence which is capable of/enables transposition and no nucleotide sequence which imparts resistance to antibiotics being present at the further gene site.

8. Coryneform bacteria according to claim 7 which produce L-lysine, wherein the coryneform bacteria belong to the genus Corynebacterium.

9. Coryneform bacteria of the genus Corynebacterium according to claim 8 which produce L-lysine, wherein these belong to the species. Corynebacterium glutamicum.

10. Coryneform bacteria according to claim 7 which produce L-lysine, wherein the copy of an open reading frame (ORF), gene or allele of lysine production is one or more of the open reading frames, genes or alleles chosen from the group consisting of accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysC^{FBR}, lysE, msiK, opcA, oxyR, ppc, ppc^{FBR}, pgk, pknA, pknB, pknD, pknG, ppsA, ptsH, ptsI, ptsM, pyc, pyc P458S, sigC, sigD, sigE, sigH, sigH, tal, thyA, tkt, tpi, zwa1, zwf and zwf A213T.

11. Coryneform bacteria according to claim 7 which produce L-lysine, wherein the copy of an open reading frame (ORF), gene or allele of lysine production is one or more of the genes or alleles chosen from the group consisting of lysC^{FBR} lysE and zwa1.

12. Coryneform bacteria according to claim 7 which produce L-lysine, wherein the copy of an open reading frame (ORF), gene or allele of lysine production is the lysE gene.

13. Coryneform bacteria according to claim 7 which produce L-lysine, wherein the copy of an open reading frame (ORF), gene or allele of lysine production is the zwa1 gene.

14. Coryneform bacteria according to claim 7 which produce L-lysine, wherein the copy of an open reading frame (ORF), gene or allele of lysine production is an lysC^{FBR} allele which codes for a feed back resistant form of aspartate kinase.

15. Coryneform bacteria according to claim 14 which produce L-lysine, wherein the feed back resistant form of aspartate kinase coded by the lysC^{FBR} allele contains an amino acid sequence according to SEQ ID NO:2, SEQ ID NO:2 having one or more amino acid exchanges chosen from the group consisting of A279T, A279V, S301F, T308I, S301Y, G345D, R320G, T311I and S381F.

16. Coryneform bacteria according to claim 14 which produce L-lysine, wherein the feed back resistant form of aspartate kinase coded by the lysC^{FBR} allele has an amino acid sequence according to SEQ ID NO:4.

17. Coryneform bacteria according to claim 14 which produce L-lysine, wherein the coding region of the lysC^{FBR} allele has the nucleotide sequence of SEQ ID NO:3.

18. Coryneform bacteria according to claim 7 which produce L-lysine, wherein the further gene site is one or more of the sites chosen from the group consisting of aecD, ccpA1, ccpA2, citA, citB, citE, fda, gluA, gluB, gluC, gluD, luxR, luxS, lysR1, lysR2, lysR3, menE, mqo, pck, pgi and poxB.

19. Coryneform bacteria according to claim 7 which produce L-lysine, wherein the further gene site is one of more of the sites chosen from the group consisting of intergenic regions of the chromosome, prophages contained in the chromosome and defective phages contained in the chromosome.

20. Processes for the preparation of one or more chemical compounds, which comprise the following steps:
a) fermentation of coryneform bacteria, which
i) instead of the singular copy of an open reading frame (ORF), gene or allele naturally present at the particular desired site (locus), have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and which
ii) optionally have at least a third copy of the said open reading frame (ORF), gene or allele at a further gene site, no nucleotide sequence which is capable of/enables episomal replication in microorganisms, no nucleotide sequence which is capable of/enables transposition and no nucleotide sequence which imparts resistance to antibiotics being present at the further gene site,
under conditions which allow expression of the said open reading frames (ORFs), genes or alleles,
b) concentration of the chemical compound(s) in the fermentation broth and/or in the cells of the bacteria,
c) isolation of the chemical compound(s), optionally
d) with constituents from the fermentation broth and/or the biomass to the extent of >(greater than) 0 to 100%.

21. Process according to claim 20, wherein the coryneform bacteria belong to the genus Corynebacterium.

22. Process according to claim 20, wherein the coryneform bacteria of the genus Corynebacterium belong to the species Corynebacterium glutamicum.

23. Process according to claim 20, wherein the chemical compound is a compound chosen from the group consisting of L-amino acids, vitamins, nucleosides and nucleotides.

24. Process according to claim 20, wherein the chemical compound is one or more L-amino acids chosen from the group consisting of L-aspartic acid, L-asparagine, L-threonine, L-serine, L-glutamic acid, L-glutamine, glycine, L-alanine, L-cysteine, L-valine, L-methionine, L-isoleucine, L-leucine, L-tyrosine, L-phenylalanine, L-histidine, L-lysine, L-tryptophan, L-proline and L-arginine.

25. Process according to claim 20, wherein the chemical compound is L-lysine.

26. Process for the preparation of L-lysine, which comprises the following steps:
a) fermentation of coryneform bacteria, which
i) instead of the singular copy of an open reading frame (ORF), gene or allele of lysine production naturally present at the particular desired site (locus), have at least two copies of the said open reading frame (ORF), gene or allele, preferably in tandem arrangement, no nucleotide sequence which is capable of/enables episomal replication in microorganisms, no nucleotide sequence which is capable of/enables transposition and no nucleotide sequence which imparts resistance to antibiotics being present at the particular site, and which optionally
ii) have at least a third copy of the said open reading frame (ORF), gene or allele of lysine production at a further gene site, no nucleotide sequence which is capable of/enables episomal replication in microorganisms, no nucleotide sequence which is capable of/enables transposition and no nucleotide sequence which imparts resistance to antibiotics being present at the further gene site,
under conditions which allow expression of the said open reading frames (ORFs), genes or alleles,
b) concentration of the L-lysine in the fermentation broth and/or in the cells of the bacteria,
c) isolation of the L-lysine, optionally
d) with constituents from the fermentation broth and/or the biomass to the extent of >(greater than) 0 to 100%.

27. Process for the preparation of L-lysine according to claim 26, wherein the coryneform bacteria belong to the genus Corynebacterium.

28. Process for the preparation of L-lysine according to claim 26, wherein the coryneform bacteria of the species Corynebacterium belong to the species Corynebacterium glutamicum.

29. Process for the preparation of L-lysine according to claim 26, wherein the copy of an open reading frame (ORF), a gene or allele of lysine production is one or more of the open reading frames, genes or alleles chosen from the group consisting of accBC, accDA, cstA, cysD, cysE, cysH, cysK, cysN, cysQ, dapA, dapB, dapC, dapD, dapE, dapF, ddh, dps, eno, gap, gap2, gdh, gnd, lysC, lysC^{FBR}, lysE, msiK, opcA, oxyR, ppc, ppc^{FBR}, pgk, pknA, pknB, pknD, pknG, ppsA, ptsH, ptsI, ptsM, pyc, pyc P458S, sigC, sigD, sigE, sigH, sigM, tal, thyA, tkt, tpi, zwa1, zwf and zwf A213T.

30. Process for the preparation of L-lysine according to claim 26, wherein the copy of an open reading frame (ORF), gene or allele of lysine production is one or more of the genes or alleles chosen from the group consisting of lysC^{FBR}, lysE and zwa1.

31. Process for the preparation of L-lysine according to claim 26, wherein the copy of an open reading frame (ORF), gene or allele of lysine production is the lysE gene.

32. Process for the preparation of L-lysine according to claim 26, wherein the copy of an open reading frame (ORF), gene or allele of lysine production is the zwa1 gene.

33. Process for the preparation of L-lysine according to claim 26, wherein the copy of an open reading frame (ORF), gene or allele of lysine production is the lysC^{FBR} allele which codes for a feed back resistant form of aspartate kinase.

34. Process for the preparation of L-lysine according to claim 33, wherein the feed back resistant form of aspartate kinase coded by the lysC^{FBR} allele contains an amino acid sequence according to SEQ ID NO:2, SEQ ID NO:2 having one or more amino acid exchanges chosen from the group consisting of A279T, A279V, S301F, T308I, S301Y, G345D, R320G, T311I and S381F.

35. Process for the preparation of L-lysine according to claim 33, wherein the feed back resistant form of aspartate kinase coded by the lysC^{FBR} allele has an amino acid sequence according to SEQ ID NO:4.

36. Process for the preparation of L-lysine according to claim 33, wherein the coding region of the lysC^{FBR} allele has the nucleotide sequence of SEQ ID NO:3.

37. Process for the preparation of L-lysine according to claim 26, wherein the further gene site is one or more of the sites chosen from the group consisting of aecD, ccpA1, ccpA2, citA, citB, citE, fda, gluA, gluB, gluC, gluD, luxR, luxS, lysR1, lysR2, lysR3, menE, mqo, pck, pgi and poxB.

38. Process for the preparation of L-lysine according to claim 26, wherein the further gene site is one of more of the sites chosen from the group consisting of intergenic regions of the chromosome, prophages contained in the chromosome and defective phages contained in the chromosome.

39. Process for the production of coryneform bacteria which produce one or more chemical compounds, wherein
a) the nucleotide sequence of a desired ORF, gene or allele, optionally including the expression and/or regulation signals, is isolated,
b) at least two copies of the nucleotide sequence of the ORF, gene or allele are arranged in a row, preferably in tandem arrangement,
c) the nucleotide sequence obtained according to b) is incorporated in a vector which does not replicate or replicates to only a limited extent in coryneform bacteria,
d) the nucleotide sequence according to b) or c) is transferred into coryneform bacteria, and
e) coryneform bacteria which have at least two copies of the desired ORF, gene or allele at the particular desired natural site instead of the singular copy of the ORF, gene or allele originally present are isolated, no nucleotide sequence which is capable of/enables episomal replication in microorganisms, no nucleotide sequence which is capable of/enables transposition and no nucleotide sequence which imparts resistance to antibiotics remaining at the particular natural site (locus), and optionally
f) at least a third copy of the open reading frame (ORF), gene or allele in question is introduced at a further gene site, no nucleotide sequence which is capable of/enables episomal replication in microorganisms, no nucleotide sequence which is capable of/enables transposition and no nucleotide sequence which imparts resistance to antibiotics remaining at the further gene site.

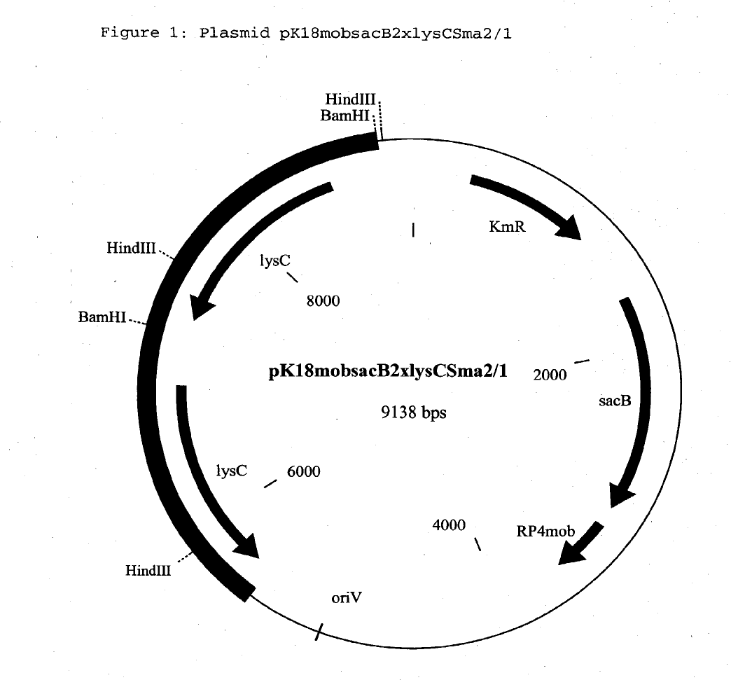

40. The plasmid pK18mobsacB2xlysCSma2/1 shown in FIG. 1 and deposited in the form of a pure culture of the strain E. coli DH5αmcr/pK18mobsacB2xlysCSma2/1 (=DH5alphamcr/pK18mobsacB2xlysCSma2/1) under number DSM14244.

41. The Corynebacterium glutamicum strain DSM13992lysC^{FBR}::lysC^{FBR} deposited in the form of a pure culture under number DSM15036.

42. The Corynebacterium glutamicum strain ATCC21513_{—}17lysE::lysE deposited in the form of a pure culture under number DSM15037.

43. The Corynebacterium glutamicum strain ATCC21513_{—}17zwa1::zwa1 deposited in the form of a pure culture under number DSM15038.

44. Coryneform bacteria according to claim 1, wherein the further gene site is selected from the group consisting of intergenic regions of the chromosome, prophages contained in the chromosome and defective phages contained in the chromosome.

45. Coryneform bacteria according to claim 44, wherein the intergenic regions are selected from table 12.

46. Coryneform bacteria according to claim 44, wherein the prophages contained in the chromosome and defective phages contained in the chromosome are selected from table 13.

47. Process according to claim 20, wherein the further gene site is selected from the group consisting of intergenic regions of the chromosome, prophages contained in the chromosome and defective phages contained in the chromosome.

48. Process according to claim 47, wherein the intergenic regions are selected from table 12.

49. Process according to claim 47, wherein the prophages contained in the chromosome and defective phages contained in the chromosome are selected from table 13.

50. Process according to claim 39, wherein the further gene site is selected from the group consisting of intergenic regions of the chromosome, prophages contained in the chromosome and defective phages contained in the chromosome.

51. Process according to claim 50, wherein the intergenic regions are selected from table 12.

52. Process according to claim 50, wherein the prophages contained in the chromosome and defective phages contained in the chromosome are selected from table 13.
